SEQUENCE LISTING (134 sequences)

SEQ ID NO: 1 - DNA, 1494 bp, Streptomyces eurythermus
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaagaggcttcgctcatgcacatggacatcgacatcgaccaggacgtctgtatcggcgccgggcagtgcgcgctggcggcaccgggcgtcttcatccaggacgacgacggctacagcaccctgctgccaggccaggagaacggcgtcaccgacccgatggcccgggaggccgcccgcgcctgcccggtcagcgccatcaccgtacgggagcgctccgcctga

SEQ ID NO: 2 - protein, 424 aa, Streptomyces eurythermus
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRTLALLGLDDPEHHRQRRLLIPSFTLKRATAQRPWIQRIVDELLDAMIARGPVADLVSAFALPVPSMVICELLGVPYADHEFFEEQSRRLLRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 3 - protein, 424 aa, Streptomyces eurythermus
  misc_feature (109)..(109): Xaa can be any naturally occurring amino acid
  misc_feature (198)..(198): Xaa can be any naturally occurring amino acid
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRXLALLGLDDPEHHRQRRLLIPSFTLKRATAQRPWIQRIVDELLDAMIARGPVADLVSAFALPVPSMVICELLGVPYADHEFFEEQSRRLXRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 4 - protein, 68 aa, Streptomyces eurythermus
MHMDIDIDQDVCIGAGQCALAAPGVFIQDDDGYSTLLPGQENGVTDPMAREAARACPVSAITVRERSA

SEQ ID NO: 5 - DNA, 1275 bp, Artificial Sequence
  seuC10M1
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgccgtctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 6 - protein, 424 aa, Artificial Sequence
  SeuC10M1
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRRLALLGLDDPEHHRQRRLLIPSFTLKRATAQRPWIQRIVDELLDAMIARGPVADLVSAFALPVPSMVICELLGVPYADHEFFEEQSRRLLRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 7 - DNA, 1275 bp, Artificial Sequence
  seuC10M2
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcgcgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 8 - protein, 424 aa, Artificial Sequence
  SeuC10M2
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRALALLGLDDPEHHRQRRLLIPSFTLKRATAQRPWIQRIVDELLDAMIARGPVADLVSAFALPVPSMVICELLGVPYADHEFFEEQSRRLLRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 9 - DNA, 1275 bp, Artificial Sequence
  seuC10M3
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgccgcggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 10 - protein, 424 aa, Artificial Sequence
  SeuC10M3
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRTLALLGLDDPEHHRQRRLLIPSFTLKRATAQRPWIQRIVDELLDAMIARGPVADLVSAFALPVPSMVICELLGVPYADHEFFEEQSRRLLRGPTGADTTDAADRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 11 - DNA, 1275 bp, Artificial Sequence
  seuC10M4
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgacgcgacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 12 - protein, 424 aa, Artificial Sequence
  SeuC10M4
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDATLALLGLDDPEHHRQRRLLIPSFTLKRATAQRPWIQRIVDELLDAMIARGPVADLVSAFALPVPSMVICELLGVPYADHEFFEEQSRRLLRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 13 - DNA, 1275 bp, Artificial Sequence
  seuC10M5
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggcgcggaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 14 - protein, 424 aa, Artificial Sequence
  SeuC10M5
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGADRTLALLGLDDPEHHRQRRLLIPSFTLKRATAQRPWIQRIVDELLDAMIARGPVADLVSAFALPVPSMVICELLGVPYADHEFFEEQSRRLLRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 15 - DNA, 1275 bp, Artificial Sequence
  seuC10M6
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccacccccgcgttcgcggccggccgcgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 16 - protein, 424 aa, Artificial Sequence
  SeuC10M6
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPAFAAGRDRTLALLGLDDPEHHRQRRLLIPSFTLKRATAQRPWIQRIVDELLDAMIARGPVADLVSAFALPVPSMVICELLGVPYADHEFFEEQSRRLLRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 17 - DNA, 1275 bp, Artificial Sequence
  seuC10M7
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgacgcgcgccgcgacggcttccccgtgcccaccccccgcttcgcggccggccgcgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 18 - protein, 424 aa, Artificial Sequence
  SeuC10M7
MKIGTTHLTRLRGLSMTELTDITGPAAQAETVAFPQDRTCPYHPPTGYDPLRDGRPLSRVTLYDGREVWLVTAQATARALLADPRLSTDARRDGFPVPTPRFAAGRDRTLALLGLDDPEHHRQRRLLIPSFTLKRATAQRPWIQRIVDELLDAMIARGPVADLVSAFALPVPSMVICELLGVPYADHEFFEEQSRRLLRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 19 - DNA, 1275 bp, Artificial Sequence
  seuC10M8
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgattcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 20 - protein, 424 aa, Artificial Sequence
  SeuC10M8
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRTLALLGLDDPEHHRQRRLLIPSFTLKRATAQRPWIQRIVDELLDAMIARGPVADLVSAFALPVPSMVICELLGVPYADHEFFEEQSRRLIRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 21 - DNA, 1275 bp, Artificial Sequence
  seuC10M9
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctggcgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 22 - protein, 424 aa, Artificial Sequence
  SeuC10M9
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRTLALLGLDDPEHHRQRRLLIPSFTLKRATAQRPWIQRIVDELLDAMIARGPVADLVSAFALPVPSMVICELLGVPYADHEFFEEQSRRLARGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 23 - DNA, 1275 bp, Artificial Sequence
  seuC10M10
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcctgctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 24 - protein, 424 aa, Artificial Sequence
  SeuC10M10
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRTLALLGLDDPEHHRQRRLLIPSFTLKRATAQRPWIQRIVDELLDAMIARGPVADLVSAFALPVPSMVICELLGVPYADHEFFEEQSRRLLRGPTGADTTDARDRLEAYLGDLIDAKAKEAEPGDGILDDLVHNRLRAGELDRSVLVSLAVLLLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 25 - DNA, 1275 bp, Artificial Sequence
  seuC10M11
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 26 - protein, 424 aa, Artificial Sequence; identical to SEQ ID NO: 2
  SeuC10M11

SEQ ID NO: 27 - DNA, 1275 bp, Artificial Sequence
  seuC10M12
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgatgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 28 - protein, 424 aa, Artificial Sequence
  SeuC10M12
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRTLALLGLDDPEHHRQRRLLIPSFTLKRATAQRPWIQRIVDELLDAMIARGPVADLVSAFALPVPSMVICELLGVPYADHEFFEEQSRRLMRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 29 - DNA, 1275 bp, Artificial Sequence
  seuC10M13
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgtttcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 30 - protein, 424 aa, Artificial Sequence
  SeuC10M13
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRTLALLGLDDPEHHRQRRLLIPSFTLKRATAQRPWIQRIVDELLDAMIARGPVADLVSAFALPVPSMVICELLGVPYADHEFFEEQSRRLFRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 31 - DNA, 1275 bp, Artificial Sequence
  seuC10M14
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctggtgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 32 - protein, 424 aa, Artificial Sequence
  SeuC10M14
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRTLALLGLDDPEHHRQRRLLIPSFTLKRATAQRPWIQRIVDELLDAMIARGPVADLVSAFALPVPSMVICELLGVPYADHEFFEEQSRRLVRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 33 - DNA, 1275 bp, Artificial Sequence
  seuC10M15
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgacccgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 34 - protein, 424 aa, Artificial Sequence
  SeuC10M15
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRTLALLGLDDPEHHRQRRLLIPSFTLKRATAQRPWIQRIVDELLDAMIARGPVADLVSAFALPVPSMVICELLGVPYADHEFFEEQSRRLTRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 35 - DNA, 1275 bp, Artificial Sequence
  seuC10M16
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgaaccgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 36 - protein, 424 aa, Artificial Sequence
  SeuC10M16
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRTLALLGLDDPEHHRQRRLLIPSFTLKRATAQRPWIQRIVDELLDAMIARGPVADLVSAFALPVPSMVICELLGVPYADHEFFEEQSRRLNRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 37 - DNA, 1275 bp, Artificial Sequence
  seuC10M17
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctggatcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 38 - protein, 424 aa, Artificial Sequence
  SeuC10M17
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRTLALLGLDDPEHHRQRRLLIPSFTLKRATAQRPWIQRIVDELLDAMIARGPVADLVSAFALPVPSMVICELLGVPYADHEFFEEQSRRLDRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 39 - DNA, 1275 bp, Artificial Sequence
  seuC10M18
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgcatcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 40 - protein, 424 aa, Artificial Sequence
  SeuC10M18
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRTLALLGLDDPEHHRQRRLLIPSFTLKRATAQRPWIQRIVDELLDAMIARGPVADLVSAFALPVPSMVICELLGVPYADHEFFEEQSRRLHRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 41 - DNA, 1275 bp, Artificial Sequence
  seuC10M19
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgtatcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 42 - protein, 424 aa, Artificial Sequence
  SeuC10M19
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRTLALLGLDDPEHHRQRRLLIPSFTLKRATAQRPWIQRIVDELLDAMIARGPVADLVSAFALPVPSMVICELLGVPYADHEFFEEQSRRLYRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 43 - DNA, 1275 bp, Artificial Sequence
  seuC10M20
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgtggcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 44 - protein, 424 aa, Artificial Sequence
  SeuC10M20
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRTLALLGLDDPEHHRQRRLLIPSFTLKRATAQRPWIQRIVDELLDAMIARGPVADLVSAFALPVPSMVICELLGVPYADHEFFEEQSRRLWRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 45 - DNA, 1275 bp, Artificial Sequence
  seuC10M21
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccacccccctgttcgcggccggccgcgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 46 - protein, 424 aa, Artificial Sequence
  SeuC10M21
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPLFAAGRDRTLALLGLDDPEHHRQRRLLIPSFTLKRATAQRPWIQRIVDELLDAMIARGPVADLVSAFALPVPSMVICELLGVPYADHEFFEEQSRRLLRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 47 - DNA, 1275 bp, Artificial Sequence
  seuC10M22
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccacccccaccttcgcggccggccgcgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 48 - protein, 424 aa, Artificial Sequence
  SeuC10M22
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPTFAAGRDRTLALLGLDDPEHHRQRRLLIPSFTLKRATAQRPWIQRIVDELLDAMIARGPVADLVSAFALPVPSMVICELLGVPYADHEFFEEQSRRLLRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 49 - DNA, 1275 bp, Artificial Sequence
  seuC10M23
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggcctggaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 50 - protein, 424 aa, Artificial Sequence
  SeuC10M23
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGLDRTLALLGLDDPEHHRQRRLLIPSFTLKRATAQRPWIQRIVDELLDAMIARGPVADLVSAFALPVPSMVICELLGVPYADHEFFEEQSRRLLRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 51 - DNA, 1275 bp, Artificial Sequence
  seuC10M24
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggcaccgaccgcacgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 52 - protein, 424 aa, Artificial Sequence
  SeuC10M24
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGTDRTLALLGLDDPEHHRQRRLLIPSFTLKRATAQRPWIQRIVDELLDAMIARGPVADLVSAFALPVPSMVICELLGVPYADHEFFEEQSRRLLRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 53 - DNA, 1275 bp, Artificial Sequence
  seuC10M25
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgctttctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 54 - protein, 424 aa, Artificial Sequence
  SeuC10M25
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRFLALLGLDDPEHHRQRRLLIPSFTLKRATAQRPWIQRIVDELLDAMIARGPVADLVSAFALPVPSMVICELLGVPYADHEFFEEQSRRLLRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 55 - DNA, 1275 bp, Artificial Sequence
  seuC10M26
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcattctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 56 - protein, 424 aa, Artificial Sequence
  SeuC10M26
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRILALLGLDDPEHHRQRRLLIPSFTLKRATAQRPWIQRIVDELLDAMIARGPVADLVSAFALPVPSMVICELLGVPYADHEFFEEQSRRLLRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 57 - DNA, 1275 bp, Artificial Sequence
  seuC10M27
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcgttctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 58 - protein, 424 aa, Artificial Sequence
  SeuC10M27
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRVLALLGLDDPEHHRQRRLLIPSFTLKRATAQRPWIQRIVDELLDAMIARGPVADLVSAFALPVPSMVICELLGVPYADHEFFEEQSRRLLRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 59 - DNA, 56 bp, Artificial Sequence
  Primer
attttgtttaactttaagaaggagatatacatatgaagatcggcacgacgcacctc

SEQ ID NO: 60 - DNA, 60 bp, Artificial Sequence
  Primer
ctacccgcagagggcggggcataagcttcctattaggcggagcgctcccgtacggtgatg

SEQ ID NO: 61 - DNA, 53 bp, Artificial Sequence
  Primer
gggtcctcaacgacaggagcacgatcatgccggaaatacaggaacgcacgctg

SEQ ID NO: 62 - DNA, 56 bp, Artificial Sequence
  Primer
ttatcgccggcatggcggccccacgggtgccggggcacaactcaatttgcgggtac

SEQ ID NO: 63 - DNA, 28 bp, Artificial Sequence
  Primer
cggccgcgaccgccgtctggccctgctc

SEQ ID NO: 64 - DNA, 28 bp, Artificial Sequence
  Primer
gagcagggccagacggcggtcgcggccg

SEQ ID NO: 65 - DNA, 26 bp, Artificial Sequence
  Primer
gccgcgaccgcgcgctggccctgctc

SEQ ID NO: 66 - DNA, 26 bp, Artificial Sequence
  Primer
gagcagggccagcgcgcggtcgcggc

SEQ ID NO: 67 - DNA, 28 bp, Artificial Sequence
  Primer
caccacggacgccgcggaccggctggag

SEQ ID NO: 68 - DNA, 28 bp, Artificial Sequence
  Primer
ctccagccggtccgcggcgtccgtggtg

SEQ ID NO: 69 - DNA, 29 bp, Artificial Sequence
  Primer
cggccggccgcgacgcgacgctggccctg

SEQ ID NO: 70 - DNA, 29 bp, Artificial Sequence
  Primer
cagggccagcgtcgcgtcgcggccggccg

SEQ ID NO: 71 - DNA, 28 bp, Artificial Sequence
  Primer
cttcgcggccggcgcggaccgcacgctg

SEQ ID NO: 72 - DNA, 28 bp, Artificial Sequence
  Primer
cagcgtgcggtccgcgccggccgcgaag

SEQ ID NO: 73 - DNA, 28 bp, Artificial Sequence
  Primer
gtgcccacccccgcgttcgcggccggcc

SEQ ID NO: 74 - DNA, 28 bp, Artificial Sequence
  Primer
ggccggccgcgaacgcgggggtgggcac

SEQ ID NO: 75 - DNA, 28 bp, Artificial Sequence
  Primer
gtccaccgacgcgcgccgcgacggcttc

SEQ ID NO: 76 - DNA, 28 bp, Artificial Sequence
  Primer
gaagccgtcgcggcgcgcgtcggtggac

SEQ ID NO: 77 - DNA, 27 bp, Artificial Sequence
  Primer
gtcccgccggctgattcgcggcccgac

SEQ ID NO: 78 - DNA, 27 bp, Artificial Sequence
  Primer
gtcgggccgcgaatcagccggcgggac

SEQ ID NO: 79 - DNA, 27 bp, Artificial Sequence
  Primer
gtcccgccggctggcgcgcggcccgac

SEQ ID NO: 80 - DNA, 27 bp, Artificial Sequence
  Primer
gtcgggccgcgcgccagccggcgggac

SEQ ID NO: 81 - DNA, 28 bp, Artificial Sequence
  Primer
gtcgctcgccgtcctgctgctggtcgcc

SEQ ID NO: 82 - DNA, 28 bp, Artificial Sequence
  Primer
ggcgaccagcagcaggacggcgagcgac

SEQ ID NO: 83 - DNA, 28 bp, Artificial Sequence
  Primer
gtcgctcgccgtcgcgctgctggtcgcc

SEQ ID NO: 84 - DNA, 28 bp, Artificial Sequence
  Primer
ggcgaccagcagcgcgacggcgagcgac

SEQ ID NO: 85 - DNA, 27 bp, Artificial Sequence
  Primer
gtcccgccggctgatgcgcggcccgac

SEQ ID NO: 86 - DNA, 27 bp, Artificial Sequence
  Primer
gtcgggccgcgcatcagccggcgggac

SEQ ID NO: 87 - DNA, 27 bp, Artificial Sequence
  Primer
gtcccgccggctgtttcgcggcccgac

SEQ ID NO: 88 - DNA, 27 bp, Artificial Sequence
  Primer
gtcgggccgcgaaacagccggcgggac

SEQ ID NO: 89 - DNA, 27 bp, Artificial Sequence
  Primer
gtcccgccggctggtgcgcggcccgac

SEQ ID NO: 90 - DNA, 27 bp, Artificial Sequence
  Primer
gtcgggccgcgcaccagccggcgggac

SEQ ID NO: 91 - DNA, 27 bp, Artificial Sequence
  Primer
gtcccgccggctgacccgcggcccgac

SEQ ID NO: 92 - DNA, 27 bp, Artificial Sequence
  Primer
gtcgggccgcgggtcagccggcgggac

SEQ ID NO: 93 - DNA, 27 bp, Artificial Sequence
  Primer
gtcccgccggctgaaccgcggcccgac

SEQ ID NO: 94 - DNA, 27 bp, Artificial Sequence
  Primer
gtcgggccgcggttcagccggcgggac

SEQ ID NO: 95 - DNA, 27 bp, Artificial Sequence
  Primer
gtcccgccggctggatcgcggcccgac

SEQ ID NO: 96 - DNA, 27 bp, Artificial Sequence
  Primer
gtcgggccgcgatccagccggcgggac

SEQ ID NO: 97 - DNA, 27 bp, Artificial Sequence
  Primer
gtcccgccggctgcatcgcggcccgac

SEQ ID NO: 98 - DNA, 27 bp, Artificial Sequence
  Primer
gtcgggccgcgatgcagccggcgggac

SEQ ID NO: 99 - DNA, 27 bp, Artificial Sequence
  Primer
gtcccgccggctgtatcgcggcccgac

SEQ ID NO: 100 - DNA, 27 bp, Artificial Sequence
  Primer
gtcgggccgcgatacagccggcgggac

SEQ ID NO: 101 - DNA, 27 bp, Artificial Sequence
  Primer
gtcccgccggctgtggcgcggcccgac

SEQ ID NO: 102 - DNA, 27 bp, Artificial Sequence
  Primer
gtcgggccgcgccacagccggcgggac

SEQ ID NO: 103 - DNA, 33 bp, Artificial Sequence
  Primer
gtgcccacccccctgttcgcggccggccgcgac

SEQ ID NO: 104 - DNA, 33 bp, Artificial Sequence
  Primer
gtcgcggccggccgcgaacaggggggtgggcac

SEQ ID NO: 105 - DNA, 33 bp, Artificial Sequence
  Primer
gtgcccacccccaccttcgcggccggccgcgac

SEQ ID NO: 106 - DNA, 33 bp, Artificial Sequence; identical to SEQ ID NO: 104
  Primer

SEQ ID NO: 107 - DNA, 28 bp, Artificial Sequence
  Primer
cttcgcggccggcctggaccgcacgctg

SEQ ID NO: 108 - DNA, 28 bp, Artificial Sequence
  Primer
cagcgtgcggtccaggccggccgcgaag

SEQ ID NO: 109 - DNA, 28 bp, Artificial Sequence
  Primer
cttcgcggccggcaccgaccgcacgctg

SEQ ID NO: 110 - DNA, 28 bp, Artificial Sequence
  Primer
cagcgtgcggtcggtgccggccgcgaag

SEQ ID NO: 111 - DNA, 26 bp, Artificial Sequence
  Primer
gccgcgaccgctttctggccctgctc

SEQ ID NO: 112 - DNA, 26 bp, Artificial Sequence
  Primer
gagcagggccagaaagcggtcgcggc

SEQ ID NO: 113 - DNA, 26 bp, Artificial Sequence
  Primer
gccgcgaccgcattctggccctgctc

SEQ ID NO: 114 - DNA, 26 bp, Artificial Sequence
  Primer
gagcagggccagaatgcggtcgcggc

SEQ ID NO: 115 - DNA, 26 bp, Artificial Sequence
  Primer
gccgcgaccgcgttctggccctgctc

SEQ ID NO: 116 - DNA, 26 bp, Artificial Sequence
  Primer
gagcagggccagaacgcggtcgcggc

SEQ ID NO: 117 - DNA, 1275 bp, Streptomyces eurythermus; identical to SEQ ID NO: 25

SEQ ID NO: 118 - DNA, 207 bp, Streptomyces eurythermus
atgcacatggacatcgacatcgaccaggacgtctgtatcggcgccgggcagtgcgcgctggcggcaccgggcgtcttcatccaggacgacgacggctacagcaccctgctgccaggccaggagaacggcgtcaccgacccgatggcccgggaggccgcccgcgcctgcccggtcagcgccatcaccgtacgggagcgctccgcctga

SEQ ID NO: 119 - protein, 454 aa, Streptomyces coelicolor
MPRPLRVAIVGSGPAGIYAADALLKSEVAADPGVSIDIFERMPAPFGLIRYGVAPDHPRIKGIITALHQVLDKPQIRLFGNVNYPTDVSLDDLRAFYDGVIFATGATADRDLSLPGIDLDGSYGAADFVAWYDGHPDFPRTWPLEAEKVAVLGVGNVALDIARVLAKTADELLPTEIPPNVYEGLKANKALEVHVFGRRGPAQAKFSPMELRELDHSPNIEVIVDPEDIDYDEGSIATRRGNKQADMVAKTLENWAIRDVGDRPHKLFLHFFESPAEILGEDGRVTGLRTERTELDGTGNVKGTGEFKDWDVQAVYRAVGYLSDQLPKLPWDLETGTVPDAGGRVVQESGEHLQSTYVTGWIRRGPIGLIGHTKGDANETVSNLLDDYANGRLQTPSSPAPEAVDAFLAERNVRFTTWDGWYRLDAAEKAQGEPHGRERVKYVEREDMLRESGA

SEQ ID NO: 120 - DNA, 1365 bp, Streptomyces coelicolor
atgccccgccctctgcgggtagccatcgtcggatccggcccggccgggatctacgccgccgacgccctgctcaagtccgaagtggccgccgaccccggtgtttccatcgacatcttcgagcgcatgcccgccccgttcggcctcatccggtacggcgtcgcgcccgaccacccgcggatcaagggcatcatcacggccctccaccaggtgctcgacaagccgcagatccgcctcttcggcaacgtgaactaccccaccgacgtcagcctggacgatctgcgcgccttctacgacggtgtgatcttcgccaccggcgccacggcggaccgggacctgtccctcccgggcatcgacctcgacggctcgtacggcgcggccgacttcgtcgcctggtacgacggccaccccgacttcccgcgcacctggccgctggaggcggagaaagtcgccgtcctcggtgtcggcaacgtcgccctggacatcgcgcgcgtcctcgccaagacggccgacgagctgctgccgaccgagatcccgccgaacgtctacgagggcctcaaggccaacaaggcgctggaggtgcacgtcttcggccgccgcggcccggcgcaggcgaagttcagcccgatggagctgcgggagctggaccactcccccaacatcgaggtgatcgtcgaccccgaggacatcgactacgacgagggctcgatcgcgacccggcgcggcaacaagcaggccgacatggtcgccaagaccctggagaactgggcgatccgcgacgtcggcgaccggccgcacaagctgttcctgcacttcttcgagtcgcccgcggagatcctcggcgaggacggcagggtgaccggcctgcgcaccgagcgcacggagctggacggcacgggcaacgtcaagggcaccggcgagttcaaggactgggacgtccaggcggtctaccgggccgtcggctacctctccgaccagctgcccaagctgccctgggacctcgagacgggcacggtcccggacgcgggcggccgggtcgtccaggagtccggcgagcacctccagtcgacgtacgtcaccggctggatccggcgcggtccgatcggcctgatcggccacaccaagggcgacgccaacgagacggtgtccaacctgctggacgactacgcgaacggccgtctccagacgccctcctcccccgctcccgaggccgtggacgcgttcctcgccgagcggaacgtccgcttcaccacctgggacggctggtaccggctcgacgccgcggagaaggcgcagggcgaaccgcacgggcgtgagcgcgtgaagtacgtcgagcgcgaggacatgctccgcgagagcggcgcctaa

SEQ ID NO: 121 - DNA, 1275 bp, Artificial Sequence
  seuC10M52
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcgatctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 122 - protein, 424 aa, Artificial Sequence
  SeuC10M52
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRDLALLGLDDPEHHRQRRLLIPSFTLKRATAQRPWIQRIVDELLDAMIARGPVADLVSAFALPVPSMVICELLGVPYADHEFFEEQSRRLLRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 123 - DNA, 1275 bp, Artificial Sequence
  seuC10M53
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgcccgctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 124 - protein, 424 aa, Artificial Sequence
  SeuC10M53
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRPLALLGLDDPEHHRQRRLLIPSFTLKRATAQRPWIQRIVDELLDAMIARGPVADLVSAFALPVPSMVICELLGVPYADHEFFEEQSRRLLRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 125 - DNA, 1275 bp, Artificial Sequence
  seuC10M30
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgctttctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgtttcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcatcctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 126 - protein, 424 aa, Artificial Sequence
  SeuC10M30
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRFLALLGLDDPEHHRQRRLLIPSFTLKRATAQRPWIQRIVDELLDAMIARGPVADLVSAFALPVPSMVICELLGVPYADHEFFEEQSRRLFRGPTGADTTDARDRLEAYLGDLIDAKAKEAEPGDGILDDLVHNRLRAGELDRSVLVSLAVILLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 127 - DNA, 1275 bp, Artificial Sequence
  seuC10M31
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgctttctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcgcgctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 128 - protein, 424 aa, Artificial Sequence
  SeuC10M31
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRFLALLGLDDPEHHRQRRLLIPSFTLKRATAQRPWIQRIVDELLDAMIARGPVADLVSAFALPVPSMVICELLGVPYADHEFFEEQSRRLLRGPTGADTTDARDRLEAYLGDLIDAKAKEAEPGDGILDDLVHNRLRAGELDRSVLVSLAVALLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 129 - DNA, 1275 bp, Artificial Sequence
  seuC10M32
atgaagatcggcacgacgcacctcactcgtctgcgaggtctttccatgacggaactgacggacatcaccggcccggctgcccaggccgaaaccgtcgcattcccccaggaccgcacctgtccctaccatccccccaccggatacgacccgctgcgcgacgggcgacccctgtcccgcgtcaccctctacgacggccgcgaggtctggctggtcaccgcccaggccaccgcccgcgccctgctcgccgacccccggctgtccaccgaccgccgccgcgacggcttccccgtgcccaccccccgcttcgcggccggccgcgaccgctttctggccctgctcgggctggacgaccccgaacaccaccggcagcgccggctgctcatcccgtcgttcaccctcaaacgcgccaccgcacagcgcccctggatccagcggatcgtcgacgaactgctggacgccatgatcgcccgggggccggtcgccgacctcgtgtccgccttcgcgctgcccgtgccgtccatggtcatctgcgaactgctcggcgtgccctacgccgaccacgagttcttcgaggaacagtcccgccggctgctgcgcggcccgaccggcgcggacaccacggacgcccgggaccggctggaggcgtacctcggcgacctgatcgacgccaaggccaaggaggccgagccgggcgacggcattctggacgacctggtccacaaccggctccgcgcgggcgagctggaccggagcgtcctggtgtcgctcgccgtcctgctgctggtcgccgggcacgagacgaccgccaacatgatctccctgggcacctacaccctgctccagcaccctgaacggctggccgagctgcgcgccgacccgtcactgctgcccgccgtcgtcgaggaactgctgcggatgctgtccatcgccgaggggctgcaacggctggcgctggaggacatcgagatcgacggcatcaccatccgggccggtgagggcgtcctcttctccacctcggtcatcaaccgggacacggccgtctacgaagaccccgacgacctggacttccaccgcgccgaccggcaccacgtggcgttcggcttcggtatccaccagtgcctgggccagaacctggcccgcgcggaactggagatcgccctcggtagcctgttcacccggctgcccgggctgcgtcttgccgccccggccgaggagatccccttcaaaccgggcgacacggtccaggggatgctggaactccccgttacctggtaa

SEQ ID NO: 130 - protein, 424 aa, Artificial Sequence
  SeuC10M32
MKIGTTHLTRLRGLSMTELTDITGPAAQAETVAFPQDRTCPYHPPTGYDPLRDGRPLSRVTLYDGREVWLVTAQATARALLADPRLSTDRRRDGFPVPTPRFAAGRDRFLALLGLDDPEHHRQRRLLIPSFTLKRATAQRPWIQRIVDELLDAMIARGPVADLVSAFALPVPSMVICELLGVPYADHEFFEEQSRRLLRGPTGADTTDARDRLEAYLGDLIDAKAKEAEPGDGILDDLVHNRLRAGELDRSVLVSLAVLLLVAGHETTANMISLGTYTLLQHPERLAELRADPSLLPAVVEELLRMLSIAEGLQRLALEDIEIDGITIRAGEGVLFSTSVINRDTAVYEDPDDLDFHRADRHHVAFGFGIHQCLGQNLARAELEIALGSLFTRLPGLRLAAPAEEIPFKPGDTVQGMLELPVTW

SEQ ID NO: 131 - DNA, 28 bp, Artificial Sequence
  Primer
cggccgcgaccgcgatctggccctgctc

SEQ ID NO: 132 - DNA, 28 bp, Artificial Sequence
  Primer
gagcagggccagatcgcggtcgcggccg

SEQ ID NO: 133 - DNA, 28 bp, Artificial Sequence
  Primer
cggccgcgaccgcccgctggccctgctc

SEQ ID NO: 134 - DNA, 28 bp, Artificial Sequence
  Primer
gagcagggccagcgggcggtcgcggccg

(19) United States
(12) Patent Application Publication
(10) Pub. No.: US 2024/0263208 A1
(43) Pub. Date: Aug. 8, 2024

(54) CYTOCHROME P450 ENZYME

(71) Applicant: HYPHA DISCOVERY LTD., Abingdon, Oxfordshire (GB)

(72) Inventors: Jonathan STEELE, Abingdon, Oxfordshire (GB); Vincent POON, Abingdon, Oxfordshire (GB); Stephen WRIGLEY, Abingdon, Oxfordshire (GB); Emily HOPKINS, Abingdon, Oxfordshire (GB); Aksana KHAN, Abingdon, Oxfordshire (GB)

(73) Assignee: HYPHA DISCOVERY LTD., Abingdon, Oxfordshire (GB)

(21) Appl. No.: 18/563,988

(22) PCT Filed: May 26, 2022
(86) PCT No.: PCT/GB2022/051349

§ 371 Date: Nov. 24, 2023

(30) Foreign Application Priority Data
May 26, 2021 (GB) 2107512.2

(51) Int. Cl. C12P 19/62; C12N 9/02

(52) U.S. Cl. CPC C12P 19/62; C12N 9/0008; C12N 9/0071; C12Y 102/07001; C12Y 114/14001

(57) ABSTRACT

The invention provides an enzyme with cytochrome P450 activity with mutations at the 109 and/or 198 positions based on P450_{SeuC10} from Streptomyces eurythermus NRRL 2539. The invention extends to related nucleic acid material and microorganisms, kits containing the enzyme, methods of producing the enzyme and methods of using the enzyme to generate oxidised organic compounds.

### FIELD OF THE INVENTION

The present invention relates to modified cytochrome P450 (CYP450) enzymes derived from a Streptomyces eurythermus NRRL 2539 CYP450. Also provided are nucleic acids encoding the enzyme, kits comprising the enzyme, and uses of the enzyme for catalysing the oxidation of organic substrates.

### BACKGROUND OF INVENTION

Cytochrome P450 (CYP) is a superfamily of haem-thiolate proteins named for the spectral absorbance peak of their carbon-monoxide bound species at 450 nm. They are found in all kingdoms of life such as animals, plants, fungi, protists, bacteria, archaea, and also in viruses.

Cytochrome P450s show extraordinary diversity in their reaction chemistry supporting the oxidative, peroxidative and reductive metabolism of a diverse range of endogenous and xenobiotic substrates.

In humans, cytochrome P450s are best known for their central role in phase I drug metabolism where they are of critical importance for two of the most significant problems in clinical pharmacology: drug-drug interactions and inter-individual variability in drug metabolism.

The most common reaction catalyzed by cytochromes P450 is a mono-oxygenase reaction. Cytochrome P450 mono-oxygenases use a haem group to oxidise molecules, often making them more water-soluble by either adding or unmasking a polar group. In general the reactions catalysed by these enzymes can be summarised as:

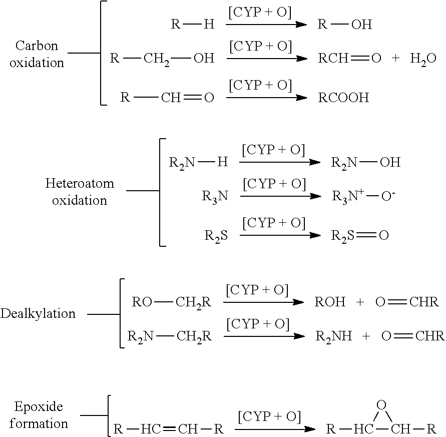

In the first line example, R—H is the substrate and R—OH is the oxygenated substrate. The oxygen is bound to the haem group in the core of the CYP enzyme, protons (H⁺) are usually indirectly derived from the reduced cofactor NADH or

NADPH via redox partner proteins, either discrete proteins or fused to the CYP, through specific amino acids in the CYP enzyme. CYP enzymes can receive electrons from a range of redox partner proteins such as cytochrome b5, a ferredoxin reductase and a ferredoxin, and adrenodoxin reductase and adrenodoxin. Bacterial P450 systems, such as that disclosed herein, utilise ferredoxin reductase and ferredoxin proteins to transfer electrons from reduced cofactor to the cytochrome P450 enzyme.

In the field of medicinal chemistry, modifications to chemical compounds are used to alter the properties of such chemical compounds. For example, tertiary butyl moieties are often used by medicinal chemists in the synthesis of drug-like molecules for introduction of hydrophobicity. However, further modifications thereof can be used to improve potency, selectivity and solubility profiles of such compounds, for example hydroxylations can be used. Hydroxylations are also the main route of metabolic degradation, another important aspect of pharmacology and medicinal chemistry. Methods for the production of these hydroxylated metabolites are sought using biotransformation with microorganisms due to being often challenging to synthesise by purely chemical means.

Whilst bacterial cytochrome P450 enzymes are known, cytochrome P450 enzymes from Actinomycete microorganisms remain relatively unreported. The induction of a cytochrome P450 in Streptomyces griseus by soybean flour (P450_{soy}) is described in Biochem and Biophys Res Comm (1986) 141, 405. Other reported examples include the isolation and properties of two forms of a P450 effecting pesticide inactivation (P450_{SU1} & _{SU2}) and two forms of 6-deoxyerythronolide B hydroxylase from Saccharopolyspora erythraea (originally classified as Streptomyces erythraeus) as described in Biochemistry (1987) 26, 6204. U.S. Pat. No. 6,884,608 describes enzymatic hydroxylation of epothilone B to epothilone F, effected with a hydroxylation enzyme produced by a strain of Amycolatopsis orientalis (originally classified as Streptomyces orientalis). A more recent example is CYP107L from Streptomyces platensis DSM40041, reported in Biotechnology and Bioengineering (2018) 115; 2156-2166 for exhibiting activities resembling some human drug metabolising P450 enzymes.

A CYP450 from S. eurythermus NRRL 2539 with particular useful capabilities for oxidising a range of organic substrates has previously been identified (co-pending application PCT/GB2020/052982). The enzyme is present in the strain Streptomyces eurythermus, a deposit of which is held by the Mycotoxin Prevention and Applied Microbiology Research Unit, National Center for Agricultural Utilization Research, Peoria, Illinois, United States of America, under the Accession number NRRL 2539. The strain has also been deposited with various other Culture Collection, with the accession numbers ATCC 14975, ATCC 19749, CBS 488.68, DSM 40014, ETH 6677, IFO 12764, IMET 43078, ISP 5014, JCM 4206, JCM 4575, RIA 1030.

The native CYP450 is referred to as P450_{SeuC10} and has the amino acid sequence set forth in SEQ ID NO: 2. The inventors have now identified certain mutants of P450_{SeuC10} which have altered regiospecificity for certain substrates, and thus have the valuable capability to produce new oxidation and hydroxylation products not generated by the wild type enzyme.

### SUMMARY OF INVENTION

It has been found that mutagenesis to alter the sequence of the enzyme shown in SEQ ID NO: 2 at residues T109 and/or L198 produces modified cytochrome P450 enzymes with improved properties including altered regiospecificity for oxidation of aliphatic terpenoid and aromatic substrates, respectively. Double mutants involving T109 and L198 are also provided. The identified mutant enzymes may be used for providing different types of oxidation reactions upon a range of organic substrates, the term “oxidation” and terms derived thereof referring to reaction types including but not limited to hydroxylation, epoxidation, carboxylation and dealkylation of the substrates. In a preferred aspect dealkylation serves to remove a methyl group, i.e., demethylation. In particular the enzymes of the invention may be used for the oxidation of a variety of aliphatic and aromatic moieties, or chemicals containing such moieties, for the purposes of C—H activation or modification of a compound's physicochemical and pharmacological properties. These mutant enzymes have not previously been identified and may be used to provide products which current enzymes are not able to produce, e.g., which may be used industrially.

Thus in a first aspect the invention provides an enzyme with cytochrome P450 activity and comprising the amino acid sequence set forth in SEQ ID NO: 3, or an amino acid sequence having at least 80% identity thereto, wherein the amino acid at the position corresponding to position 109 of SEQ ID NO: 3 is not threonine, and/or the amino acid at the position corresponding to position 198 of SEQ ID NO: 3 is not leucine. Preferred enzymes are as set out in the Examples.

In a second aspect the invention provides a nucleic acid molecule encoding an enzyme of the invention with cytochrome P450 activity.

In a third aspect the invention provides a recombinant construct comprising a nucleic acid molecule of the invention operatively linked to an expression control sequence.

In a fourth aspect the invention provides a vector comprising a nucleic acid molecule of the invention or a recombinant construct of the invention.

In a fifth aspect the invention provides a microorganism comprising a nucleic acid molecule of the invention, a recombinant construct of the invention or a vector of the invention.

In a sixth aspect the invention provides a kit comprising:
i) an enzyme of the invention with cytochrome P450 activity; and/or
ii) a microorganism that expresses an enzyme of the invention with cytochrome P450 activity, preferably a microorganism of the invention, or a lysate thereof; and/or
iii) a nucleic acid molecule of the invention, a recombinant construct of the invention or a vector of the invention.
   In a seventh aspect the invention provides a method of oxidising an organic compound, comprising contacting the organic compound with an enzyme of the invention. Preferably the oxidised organic compound is harvested, collected, isolated, enriched, purified and/or processed.

In an eighth aspect the invention provides use of the enzyme of the invention to oxidise an organic compound.

In a ninth aspect the invention provides a method of producing an enzyme of the invention with cytochrome P450 activity, comprising introducing a nucleic acid molecule of the invention, a recombinant construct of the invention or a vector of the invention into a microorganism, and expressing the cytochrome P450 enzyme in the microorganism, and optionally purifying the enzyme.

### BRIEF DESCRIPTION OF THE FIGURES

The Invention is Described with Reference to the Accompanying Drawings, Wherein:

FIG. 1 shows schematic examples of biotransformations effected by the use of the cytochrome P450 enzyme comprising SEQ ID NO: 2 of the present invention. FIG. 1(a) shows epoxidation and hydroxylation of carbamazepine; FIG. 1(b) shows hydroxylation of bosentan (with demethylation as a minor side reaction); FIG. 1(c) shows hydroxylation of diclofenac; FIG. 1(d) shows hydroxylation of a methyl group of meloxicam, with some further oxidation to yield a carboxyl moiety; FIG. 1(e) shows hydroxylation of tivantinib; and FIG. 1(f) shows hydroxylation of ambroxide to produce multiple products with some further oxidation to produce a ketone; and FIG. 1(g) shows selective hydroxylation of buparvaquone.

FIG. 2 shows the expression plasmid pHD05-SeuC10-SeuF08 from which the native CYP450 SeuC10 (SEQ ID NO: 2) and its associated ferredoxin SeuF08 (SEQ ID NO: 4) are expressed.

FIG. 3 shows the carbon monoxide difference spectra of crude enzyme extracts containing P450_{SeuC10} protein and selected variants. Samples were prepared from IPTG-induced cultures of E. coli Tuner (DE3) cells containing the relevant expression plasmid. FIG. 3(a) shows the carbon monoxide difference spectrum of crude extract containing P450_{SeuC10} (SEQ ID NO: 2); FIG. 3(b) shows the carbon monoxide difference spectrum of crude extract containing P450_{SeuC10} with L198A mutation (SEQ ID NO: 22); FIG. 3(c) shows the carbon monoxide difference spectrum of crude extract containing P450_{SeuC10} with L198F mutation (SEQ ID NO: 30) and FIG. 3(d) shows the carbon monoxide difference spectrum of crude extract containing P450_{SeuC10} with L198W mutation (SEQ ID NO: 44). Similar results were observed with crude extracts containing P450_{SeuC10} with T109F mutation (SEQ ID NO: 54), L198F and T109F double mutations (SEQ ID NO: 126), T109F and 1259L double mutations (SEQ ID NO: 130), and T109P mutation (SEQ ID NO: 124) (data not shown).

FIG. 4 show UPLC-MS chromatograms of various reactions performed at the 100 μL screening scale:

FIG. 4(a) shows chromatograms of post-reaction extracts using lyophilised material of recombinant E. coli expressing P450_{SeuC10} (SEQ ID NO: 2), ferredoxin_{SeuF08} and ferredoxin reductase_{SCF15A} (left panel) and the T109F mutant of P450_{SeuC10} (SEQ ID NO: 54), ferredoxin_{SeuF08} and ferredoxin reductase_{SCF15A} (right panel). The top chromatograms show EIC_{237m/z} (ambroxide 2.22 mins) and bottom chromatograms show EIC_{253m/z} (oxidised ambroxide products at 1.51, 1.54, 1.61 and 2.04 mins).

FIG. 4(b) shows chromatograms of post-reaction extracts using lyophilised material of recombinant E. coli expressing P450_{SeuC10} (SEQ ID NO: 2), ferredoxin_{SeuF08} and ferredoxin reductase_{SCF15A} (left panel) and the L198F mutant of P450_{SeuC10} (SEQ ID NO: 30), ferredoxin_{SeuF08} and ferredoxin reductase_{SCF15A} (right panel). The top to bottom chromatograms show UV₂₃₀ₙₘ, EIC_{237m/z} (carbamazepine 1.31 mins) and EIC_{253m/z} (carbamazepine epoxide 1.14 mins and new oxidised carbamazepine product, 2-hydroxycarbamazepine at 1.18 mins), respectively.

FIG. 4(c) shows chromatograms of post-reaction extracts using lyophilised material of recombinant E. coli expressing P450_{SeuC10} (SEQ ID NO: 2), ferredoxin_{SeuF08} and ferredoxin reductase_{SCF15A} (left panel) and the L198W mutant of P450_{SeuC10} (SEQ ID NO: 44), ferredoxin_{SeuF08} and ferredoxin reductase_{SCF15A}. The top to bottom chromatograms show UV₂₃₀ₙₘ, EIC_{237m/z} (carbamazepine ˜1.31 mins) and EIC_{253m/z} (carbamazepine epoxide 1.14 mins and new oxidised carbamazepine product, 2-hydroxycarbamazepine at 1.18 mins), respectively.

FIG. 5 shows a UPLC-MS chromatogram of a post-reaction extract using lyophilised material of recombinant E. coli expressing the T109F/1259L mutant of P450_{SeuC10} (SEQ ID NO: 130, M32), ferredoxin_{SeuF08} and ferredoxin reductase_{SCF15A} on clarithromycin. The top and bottom chromatograms on the left show EIC_{748m/z} (clarithromycin at 3.16 mins) and EIC_{734m/z} (3″-O-demethylclarithromycin at 3.11 mins), respectively. The right-hand side of the figure shows the positive ion ESI mass spectra of the peaks at 3.16 mins (top, clarithromycin) and 3.11 mins (bottom, 3″-O-demethylclarithromycin). Finally, the figure shows the structure of clarithromycin and this new product, which is produced by reaction with the enzyme (corresponding to 3.11 minutes on the chromatogram).

FIG. 6 shows the UPLC-MS chromatograms of post-reaction extracts using lyophilised material of recombinant E. coli expressing the L198T (M15), T109F/1259A (M31) and T109F/1259L (M32) mutants of P450_{SeuC10} (SEQ ID NOs: 34, 128 and 130, respectively) and the wild-type (WT) enzyme (SEQ ID NO: 2), ferredoxin_{SeuF08} and ferredoxin reductase_{SCF15A} on clarithromycin. The chromatograms show EIC_{748+734m/z} (the chromatogram for M32 is the only one to show the presence of the new 3″-O-demethylclarithromycin product at 3.12 mins in clearly detectable yield).

FIG. 7 shows the UPLC-MS chromatograms of post-reaction extracts using lyophilised material of recombinant E. coli expressing P450_{SeuC10} (SEQ ID NO: 2), ferredoxin_{SeuF08} and ferredoxin reductase_{SCF15A} (lower panel) and the T109F/1259L mutant of P450_{SeuC10} (M32, SEQ ID NO: 130, upper panel), ferredoxin_{SeuF08} and ferredoxin reductase_{SCF15A} on sclareol. The chromatograms show EIC_{273+289m/z} (sclareol 2.04 mins, oxidised products at 1.42, 1.55 and 1.88 mins).

FIG. 8 shows the UPLC-MS chromatograms of post-reaction extracts using lyophilised material of recombinant E. coli expressing P450_{SeuC10} (SEQ ID NO: 2), ferredoxin_{SeuF08} and ferredoxin reductase_{SCF15A} (lower panel) and the T109P mutant of P450_{SeuC10} (M53, SEQ ID NO: 124, upper panel), ferredoxin_{SeuF08} and ferredoxin reductase_{SCF15A} on buparvaquone. The chromatograms show UV₂₈₂ₙₘ (buparvaquone 2.46 mins, oxidised product at 1.97 mins in improved yield in the top chromatogram).

FIG. 9 shows the UPLC-MS chromatograms of post-reaction extracts using lyophilised material of recombinant E. coli expressing P450_{SeuC10} (SEQ ID NO: 2), ferredoxin_{SeuF08} and ferredoxin reductase_{SCF15A} (lower panel) and the T109P mutant of P450_{SeuC10} (M53, SEQ ID NO: 124, upper panel), ferredoxin_{SeuF08} and ferredoxin reductase_{SCF15A} on ebastine. The chromatograms show UV₂₅₄ₙₘ (ebastine 1.57 mins, oxidised product at 1.38 mins in improved yield in the upper chromatogram).

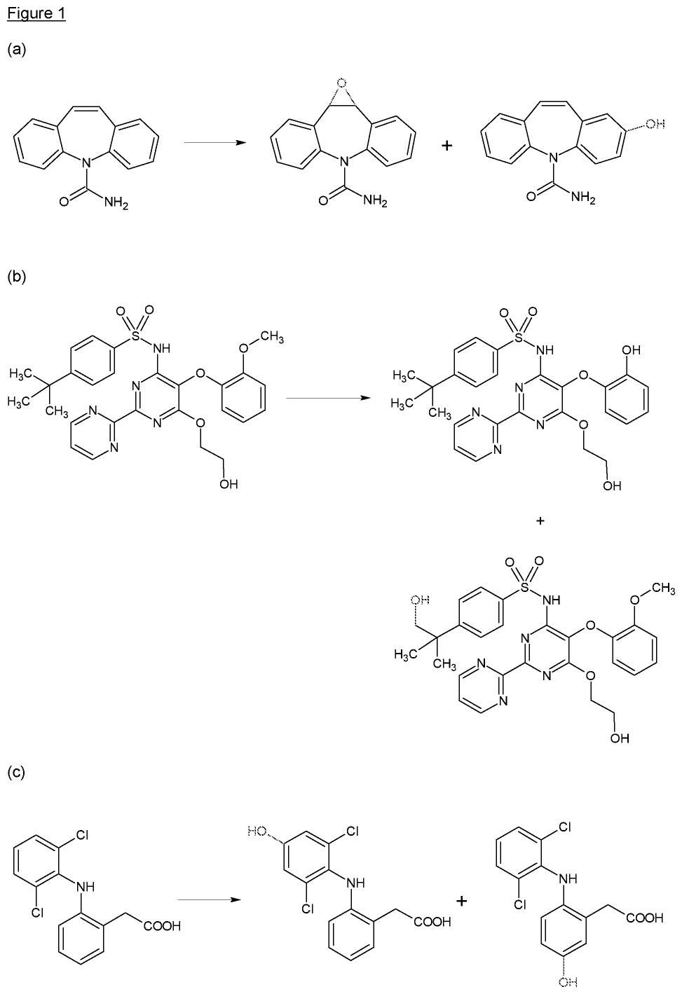

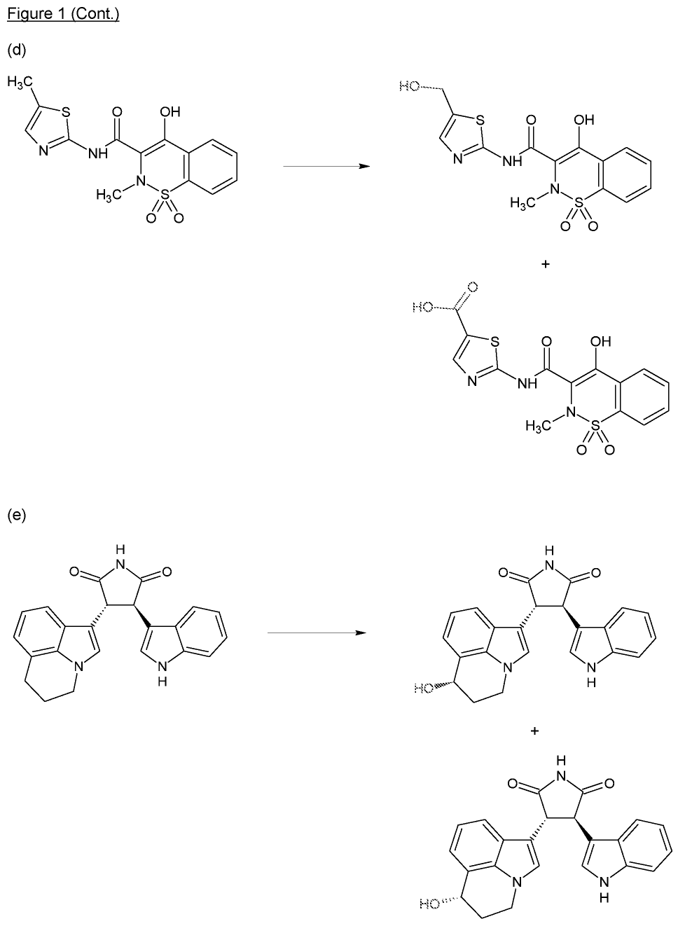

### DESCRIPTION OF INVENTION

The first aspect of the invention provides an enzyme with cytochrome P450 activity and comprising the amino acid sequence set forth in SEQ ID NO: 3, or an amino acid sequence having at least 80% identity thereto, wherein the amino acid at the position corresponding to position 109 of SEQ ID NO: 3 is not threonine, and/or the amino acid at the position corresponding to position 198 of SEQ ID NO: 3 is not leucine.

As used herein the terms “enzyme with cytochrome P450 activity” and “cytochrome P450 enzyme” are used interchangeably. The cytochrome P450 activity of a protein (e.g., a cytochrome P450 enzyme or putative cytochrome P450 enzyme) may be measured using standard methods in the art, as described further below. The term “enzyme” as used herein has its standard meaning in the art, i.e., it refers to a protein (or polypeptide) catalyst. Thus an enzyme of the invention is a polypeptide.

The amino acid sequence of SEQ ID NO: 3 corresponds to the wild type sequence of P450_{SeuC10} with unspecified residues at positions 109 and 198. In the wild type sequence (set forth in SEQ ID NO: 2), the amino acid at position 109 is threonine and the amino acid at position 198 is leucine. As detailed above, in the enzyme of the invention, the amino acid at the position corresponding to position 109 of SEQ ID NO: 3 is not threonine, and/or the amino acid at the position corresponding to position 198 of SEQ ID NO: 3 is not leucine. That is to say, in one embodiment the amino acid at the position corresponding to position 109 of SEQ ID NO: 3 is threonine, and the amino acid at the position corresponding to position 198 of SEQ ID NO: 3 is not leucine; in another embodiment the amino acid at the position corresponding to position 109 of SEQ ID NO: 3 is not threonine, and the amino acid at the position corresponding to position 198 of SEQ ID NO: 3 is leucine; in a third embodiment the amino acid at the position corresponding to position 109 of SEQ ID NO: 3 is not threonine, and the amino acid at the position corresponding to position 198 of SEQ ID NO: 3 is not leucine.

The position in an enzyme of the invention “corresponding to position 109 of SEQ ID NO: 3” is the position in the enzyme of the invention which aligns to position 109 of SEQ ID NO: 3 (i.e., is equivalent to position 109 of SEQ ID NO: 3), when a sequence alignment is performed of the sequence of the enzyme and SEQ ID NO: 3. This position may or may not be position 109 of the enzyme of the invention, depending on the nature of any variance in the sequences of the enzyme of the invention and SEQ ID NO: 3. Similarly, the position in an enzyme of the invention “corresponding to position 198 of SEQ ID NO: 3” is the position in the enzyme of the invention which aligns to position 198 of SEQ ID NO: 3 (i.e., is equivalent to position 198 of SEQ ID NO: 3), when a sequence alignment is performed of the sequence of the enzyme and SEQ ID NO: 3. Again, this position may or may not be position 198 of the enzyme of the invention, depending on the nature of any variance in the sequences of the enzyme of the invention and SEQ ID NO: 3.

Sequence alignments may be performed using any convenient method. Generally a computer programme capable of performing a pairwise or multiple sequence alignment is used. Suitable alignment programmes include EMBOSS Needle or EMBOSS stretcher (both Rice, P. et al., Trends Genet. 16, (6) pp. 276-277, 2000) which may be used for pairwise sequence alignments, while Clustal Omega (Sievers F et al., Mol. Syst. Biol. 7:539, 2011) or MUSCLE (Edgar, R. C., Nucleic Acids Res. 32(5): 1792-1797, 2004) may be used for multiple sequence alignments, though any other appropriate programme may be used. Whether the alignment is pairwise or multiple, it must be performed globally (i.e., across the entirety of the sequences in question) rather than locally.

Sequence alignments may be performed using for instance standard Clustal Omega parameters: matrix Gonnet, gap opening penalty 6, gap extension penalty 1. Alternatively the standard EMBOSS Needle parameters may be used: matrix BLOSUM62, gap opening penalty 10, gap extension penalty 0.5. Any other suitable parameters may alternatively be used.

For the purposes of the present invention, where there is dispute between sequence alignments obtained by different methods, the value obtained by global pairwise alignment using EMBOSS Needle with default parameters shall be considered valid.

As detailed above, the enzyme of the invention comprises an amino acid sequence with at least 80% identity to SEQ ID NO: 3 (subject to the requirements regarding the amino acids at the positions corresponding to 109 and 198 of SEQ ID NO: 3). Preferably, the enzyme of the invention comprises an amino acid sequence with at least 85%, 90%, 95%, 96%, 97%, 98% or 99% identity to SEQ ID NO: 3 (subject to the requirements regarding the amino acids at the positions corresponding to 109 and 198 of SEQ ID NO: 3). In another preferred embodiment the enzyme of the invention comprises the amino acid sequence of SEQ ID NO: 3 (subject to the requirements regarding the amino acids at the positions corresponding to 109 and 198 of SEQ ID NO: 3). In other embodiments, the enzyme of the invention may consist of the amino acid sequences described above. Preferably the enzyme is a non-native enzyme with a non-native sequence, i.e., is not found in nature.

Sequence identity may be assessed by any convenient method. Generally, it is determined by performing a sequence alignment as described above. As detailed above, to calculate percentage sequence identity any alignment must be performed globally, and where there is dispute between sequence alignments obtained by different methods, the value obtained by global pairwise alignment using EMBOSS Needle with default parameters shall be considered valid.

When the amino acid at the position corresponding to position 109 of SEQ ID NO: 3 is not threonine, it may be any amino acid other than threonine. Preferably it is a proteinogenic amino acid encoded by the genetic code, though it may be a proteinogenic amino acid not encoded by the genetic code, or a non-proteinogenic amino acid. In a particular embodiment the amino acid at the position corresponding to position 109 of SEQ ID NO: 3 is a hydrophobic amino acid or an aromatic amino acid (which are as defined hereinafter). Preferably the amino acid at the position corresponding to position 109 of SEQ ID NO: 3 is selected from alanine, valine and phenylalanine. In a further alternative the residue may be proline. Most preferably the amino acid at the position corresponding to position 109 of SEQ ID NO: 3 is phenylalanine.

Similarly, when the amino acid at the position corresponding to position 198 of SEQ ID NO: 3 is not leucine, it may be any amino acid other than leucine. Preferably it is a proteinogenic amino acid encoded by the genetic code, though it may be a proteinogenic amino acid not encoded by the genetic code, or a non-proteinogenic amino acid. In a particular embodiment the amino acid at the position corresponding to position 198 of SEQ ID NO: 3 is a hydrophobic amino acid, or an aromatic amino acid. Most preferably the amino acid at the position corresponding to position 198 of SEQ ID NO: 3 is alanine, phenylalanine or tryptophan.

In a particular embodiment, the enzyme of the invention has a phenylalanine residue at the position corresponding to position 109 of SEQ ID NO: 3 and a leucine residue at the position corresponding to position 198 of SEQ ID NO: 3. The amino acid sequence corresponding to SEQ ID NO: 3 with a phenylalanine residue at position 109 and a leucine residue at position 198 is set forth in SEQ ID NO: 54. The enzyme of SEQ ID NO: 54 is annotated as P450_{SeuC10} M25 in the examples. Thus in a particular embodiment the enzyme of the invention comprises the amino acid sequence set forth in SEQ ID NO: 54, or an amino acid sequence with at least 80% identity to SEQ ID NO: 54. In preferred embodiments, the enzyme of the invention comprises an amino acid sequence with at least 85%, 90%, 95%, 96%, 97%, 98% or 99% identity to SEQ ID NO: 54. In another preferred embodiment the enzyme of the invention comprises the amino acid sequence set forth in SEQ ID NO: 54. In other embodiments, the enzyme of the invention may consist of the amino acid sequences described above.

When the enzyme of the invention is or comprises a variant of SEQ ID NO: 54 (i.e., an enzyme having at least 80% but less than 100% sequence identity to SEQ ID NO: 54) the amino acid at the position corresponding to position 109 of SEQ ID NO: 54 (i.e., the position corresponding to position 109 of SEQ ID NO: 3) is phenylalanine.

In another embodiment, the enzyme of the invention has an alanine residue at the position corresponding to position 109 of SEQ ID NO: 3 and a leucine residue at the position corresponding to position 198 of SEQ ID NO: 3. The amino acid sequence corresponding to SEQ ID NO: 3 with an alanine residue at position 109 and a leucine residue at position 198 is set forth in SEQ ID NO: 8. The enzyme of SEQ ID NO: 8 is annotated as P450_{SeuC10} M2 in the examples. Thus in a particular embodiment the enzyme of the invention comprises the amino acid sequence set forth in SEQ ID NO: 8, or an amino acid sequence with at least 80% identity to SEQ ID NO: 8. In preferred embodiments, the enzyme of the invention comprises an amino acid sequence with at least 85%, 90%, 95%, 96%, 97%, 98% or 99% identity to SEQ ID NO: 8. In another preferred embodiment the enzyme of the invention comprises the amino acid sequence set forth in SEQ ID NO: 8. In other embodiments, the enzyme of the invention may consist of the amino acid sequences described above.

When the enzyme of the invention is or comprises a variant of SEQ ID NO: 8 (i.e., an enzyme having at least 80% but less than 100% sequence identity to SEQ ID NO: 8) the amino acid at the position corresponding to position 109 of SEQ ID NO: 8 (i.e., the position corresponding to position 109 of SEQ ID NO: 3) is alanine.

In another embodiment, the enzyme of the invention has a valine residue at the position corresponding to position 109 of SEQ ID NO: 3 and a leucine residue at the position corresponding to position 198 of SEQ ID NO: 3. The amino acid sequence corresponding to SEQ ID NO: 3 with a valine residue at position 109 and a leucine residue at position 198 is set forth in SEQ ID NO: 58. The enzyme of SEQ ID NO: 58 is annotated as P450_{SeuC10} M27 in the examples. Thus in a particular embodiment the enzyme of the invention comprises the amino acid sequence set forth in SEQ ID NO: 58, or an amino acid sequence with at least 80% identity to SEQ ID NO: 58. In preferred embodiments, the enzyme of the invention comprises an amino acid sequence with at least 85%, 90%, 95%, 96%, 97%, 98% or 99% identity to SEQ ID NO: 58. In another preferred embodiment the enzyme of the invention comprises the amino acid sequence set forth in SEQ ID NO: 58. In other embodiments, the enzyme of the invention may consist of the amino acid sequences described above.

When the enzyme of the invention is or comprises a variant of SEQ ID NO: 58 (i.e., an enzyme having at least 80% but less than 100% sequence identity to SEQ ID NO: 58) the amino acid at the position corresponding to position 109 of SEQ ID NO: 58 (i.e., the position corresponding to position 109 of SEQ ID NO: 3) is valine.

In another embodiment, the enzyme of the invention has a proline residue at the position corresponding to position 109 of SEQ ID NO: 3 and a leucine residue at the position corresponding to position 198 of SEQ ID NO: 3. The amino acid sequence corresponding to SEQ ID NO: 3 with a proline residue at position 109 and a leucine residue at position 198 is set forth in SEQ ID NO: 124. The enzyme of SEQ ID NO: 124 is annotated as P450_{SeuC10} M53 in the examples. Thus in a particular embodiment the enzyme of the invention comprises the amino acid sequence set forth in SEQ ID NO: 124, or an amino acid sequence with at least 80% identity to SEQ ID NO: 124. In preferred embodiments, the enzyme of the invention comprises an amino acid sequence with at least 85%, 90%, 95%, 96%, 97%, 98% or 99% identity to SEQ ID NO: 124. In another preferred embodiment the enzyme of the invention comprises the amino acid sequence set forth in SEQ ID NO: 124. In other embodiments, the enzyme of the invention may consist of the amino acid sequences described above.

When the enzyme of the invention is or comprises a variant of SEQ ID NO: 124 (i.e., an enzyme having at least 80% but less than 100% sequence identity to SEQ ID NO: 124) the amino acid at the position corresponding to position 109 of SEQ ID NO: 124 (i.e., the position corresponding to position 109 of SEQ ID NO: 3) is proline.

In another embodiment, the enzyme of the invention has a threonine residue at the position corresponding to position 109 of SEQ ID NO: 3 and an alanine residue at the position corresponding to position 198 of SEQ ID NO: 3. The amino acid sequence corresponding to SEQ ID NO: 3 with a threonine residue at position 109 and an alanine residue at position 198 is set forth in SEQ ID NO: 22. The enzyme of SEQ ID NO: 22 is annotated as P450_{SeuC10} M9 in the examples. Thus in a particular embodiment the enzyme of the invention comprises the amino acid sequence set forth in SEQ ID NO: 22, or an amino acid sequence with at least 80% identity to SEQ ID NO: 22. In preferred embodiments, the enzyme of the invention comprises an amino acid sequence with at least 85%, 90%, 95%, 96%, 97%, 98% or 99% identity to SEQ ID NO: 22. In another preferred embodiment the enzyme of the invention comprises the amino acid sequence set forth in SEQ ID NO: 22. In other embodiments, the enzyme of the invention may consist of the amino acid sequences described above.

When the enzyme of the invention is or comprises a variant of SEQ ID NO: 22 (i.e., an enzyme having at least 80% but less than 100% sequence identity to SEQ ID NO: 22) the amino acid at the position corresponding to position 198 of SEQ ID NO: 22 (i.e., the position corresponding to position 198 of SEQ ID NO: 3) is alanine.

In another embodiment, the enzyme of the invention has a threonine residue at the position corresponding to position 109 of SEQ ID NO: 3 and a phenylalanine residue at the position corresponding to position 198 of SEQ ID NO: 3. The amino acid sequence corresponding to SEQ ID NO: 3 with a threonine residue at position 109 and a phenylalanine residue at position 198 is set forth in SEQ ID NO: 30. The enzyme of SEQ ID NO: 30 is annotated as P450_{SeuC10} M13 in the examples. Thus in a particular embodiment the enzyme of the invention comprises the amino acid sequence set forth in SEQ ID NO: 30, or an amino acid sequence with at least 80% identity to SEQ ID NO: 30. In preferred embodiments, the enzyme of the invention comprises an amino acid sequence with at least 85%, 90%, 95%, 96%, 97%, 98% or 99% identity to SEQ ID NO: 30. In another preferred embodiment the enzyme of the invention comprises the amino acid sequence set forth in SEQ ID NO: 30. In other embodiments, the enzyme of the invention may consist of the amino acid sequences described above.

When the enzyme of the invention is or comprises a variant of SEQ ID NO: 30 (i.e., an enzyme having at least 80% but less than 100% sequence identity to SEQ ID NO: 30) the amino acid at the position corresponding to position 198 of SEQ ID NO: 30 (i.e., the position corresponding to position 198 of SEQ ID NO: 3) is phenylalanine.

In another embodiment, the enzyme of the invention has a threonine residue at the position corresponding to position 109 of SEQ ID NO: 3 and a tryptophan residue at the position corresponding to position 198 of SEQ ID NO: 3. The amino acid sequence corresponding to SEQ ID NO: 3 with a threonine residue at position 109 and a tryptophan residue at position 198 is set forth in SEQ ID NO: 44. The enzyme of SEQ ID NO: 44 is annotated as P450_{SeuC10} M20 in the examples. Thus in a particular embodiment the enzyme of the invention comprises the amino acid sequence set forth in SEQ ID NO: 44, or an amino acid sequence with at least 80% identity to SEQ ID NO: 44. In preferred embodiments, the enzyme of the invention comprises an amino acid sequence with at least 85%, 90%, 95%, 96%, 97%, 98% or 99% identity to SEQ ID NO: 44. In another preferred embodiment the enzyme of the invention comprises the amino acid sequence set forth in SEQ ID NO: 44. In other embodiments, the enzyme of the invention may consist of the amino acid sequences described above.

When the enzyme of the invention is or comprises a variant of SEQ ID NO: 44 (i.e., an enzyme having at least 80% but less than 100% sequence identity to SEQ ID NO: 44) the amino acid at the position corresponding to position 198 of SEQ ID NO: 44 (i.e., the position corresponding to position 198 of SEQ ID NO: 3) is tryptophan.

In particular embodiments the enzyme of the invention may comprise combinations of the amino acid substitutions at the positions corresponding to positions 109 and 198 of SEQ ID NO: 3, as described above. For instance, the enzyme of the invention may comprise an amino acid selected from alanine, phenylalanine and valine (or proline) at the position corresponding to position 109 of SEQ ID NO: 3 (most preferably a phenylalanine residue) and an amino acid selected from alanine, phenylalanine and tryptophan at the position corresponding to position 198 of SEQ ID NO: 3.

Thus, the enzyme of the invention may comprise more than one mutation, i.e., the enzyme may comprise a mutation at both positions 109 and 198 or may comprise one or more additional mutations beyond the mutations at positions 109 and/or 198.

The invention therefore provides, in another embodiment, an enzyme of the invention, wherein
a) the amino acid at the position corresponding to position 109 of SEQ ID NO: 3 is not threonine, and
b) the amino acid at the position corresponding to position 259 of SEQ ID NO: 3 is not isoleucine and/or the amino acid at the position corresponding to position 198 of SEQ ID NO: 3 is not leucine. Preferably the amino acid at the position corresponding to position 109 of SEQ ID NO: 3 is phenylalanine.

In particularly preferred aspects, in the enzyme of the invention,
a) the amino acid at the position corresponding to position 109 of SEQ ID NO: 3 is phenylalanine and the amino acid at the position corresponding to position 259 of SEQ ID NO: 3 is leucine; or
b) the amino acid at the position corresponding to position 109 of SEQ ID NO: 3 is phenylalanine and the amino acid at the position corresponding to position 198 of SEQ ID NO: 3 is phenylalanine.

The amino acid sequence corresponding to SEQ ID NO: 3 with a phenylalanine residue at position 109 and a leucine residue at position 259 is set forth in SEQ ID NO: 130. The enzyme of SEQ ID NO: 130 is annotated as P450_{SeuC10} M32 in the examples. The amino acid sequence corresponding to SEQ ID NO: 3 with a phenylalanine residue at position 109 and a phenylalanine residue at position 198 is set forth in SEQ ID NO: 126. The enzyme of SEQ ID NO: 126 is annotated as P450_{SeuC10} M30 in the examples. Thus in a particular embodiment the enzyme of the invention comprises the amino acid sequence set forth in SEQ ID NO: 126 or 130, or an amino acid sequence with at least 80% identity to SEQ ID NO: 126 or 130. In preferred embodiments, the enzyme of the invention comprises an amino acid sequence with at least 85%, 90%, 95%, 96%, 97%, 98% or 99% identity to SEQ ID NO: 126 or 130. In another preferred embodiment the enzyme of the invention comprises the amino acid sequence set forth in SEQ ID NO: 126 or 130. In other embodiments, the enzyme of the invention may consist of the amino acid sequences described above.

When the enzyme of the invention is or comprises a variant of SEQ ID NO: 126 or 130 (i.e., an enzyme having at least 80% but less than 100% sequence identity to SEQ ID NO: 126 or 130) the amino acid at the position corresponding to position 109 of SEQ ID NO: 126 or 130 (i.e., the position corresponding to position 109 of SEQ ID NO: 3) is phenylalanine and the amino acid at the position corresponding to position 259 of SEQ ID NO: 130 is leucine and the amino acid at the position corresponding to position 198 of SEQ ID NO: 126 is phenylalanine.

In an alternative embodiment, the invention also provides an enzyme of the invention, wherein
a) the amino acid at the position corresponding to position 198 of SEQ ID NO: 3 is not leucine, and
b) the amino acid at the position corresponding to position 109 of SEQ ID NO: 3 is not threonine. Preferably the amino acid at the position corresponding to position 198 of SEQ ID NO: 3 is phenylalanine.

In particularly preferred aspects, in the enzyme of the invention,
a) the amino acid at the position corresponding to position 198 of SEQ ID NO: 3 is phenylalanine, and
b) the amino acid at the position corresponding to position 109 of SEQ ID NO: 3 is phenylalanine.

The amino acid sequence corresponding to SEQ ID NO: 3 with a phenylalanine residue at positions 109 and 198 is set forth in SEQ ID NO: 126, which is as described hereinbefore.

In embodiments of the invention where the enzyme comprises an amino acid sequence which is a variant of SEQ ID NO: 3 (or any of the specific mutant sequences detailed above), i.e., a sequence with at least 80% but less than 100% identity to SEQ ID NO: 3 (or any of the specific mutant sequences detailed above), that variant may be altered relative to the specified sequence by substitution, addition and/or deletion of amino acid residues. When the enzyme sequence is modified by substitution of a particular amino acid residue, the substitution may be a conservative amino acid substitution. The term “conservative amino acid substitution”, as used herein, refers to an amino acid substitution in which one amino acid residue is replaced with another amino acid residue having a similar side chain. Amino acids with similar side chains tend to have similar properties, and thus a conservative substitution of an amino acid important for the structure or function of a polypeptide may be expected to affect polypeptide structure/function less than a non-conservative amino acid substitution at the same position. Preferred variants have the same functional properties as the parent mutant enzyme, i.e., generate the same products from a particular substrate. Families of amino acid residues having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine), non-polar/hydrophobic side chains (e.g., glycine, cysteine, alanine, valine, leucine, isoleucine, proline, phenylalanine, methionine, tryptophan) and aromatic side chains (e.g., tyrosine, phenylalanine, tryptophan, histidine). Thus a conservative amino acid substitution may be considered to be a substitution in which a particular amino acid residue is substituted for a different amino acid in the same family. However, amino acid substitutions may equally be non-conservative, with one amino acid substituted for another with a side-chain belonging to a different family.

Amino acid substitutions or additions in the scope of the invention may be made using a proteinogenic amino acid encoded by the genetic code, a proteinogenic amino acid not encoded by the genetic code, or a non-proteinogenic amino acid. Preferably any amino acid substitution or addition is made using a proteinogenic amino acid. Substitutions and additions may be made using amino acids which do not occur naturally, but which are modifications of amino acids which occur naturally. For example derivatives of amino acids such as methylated amino acids may be used.

The enzyme of the invention may be synthesised by any method known in the art. In particular, the specific binding molecule may be synthesised using a protein expression system, such as a cellular expression system, particularly a cellular expression system using prokaryotic cells. Preferably a cellular expression system for expression of the enzyme of the invention uses bacterial cells. Escherichia coli cells may be particularly suitable. Culture using methods known in the art may be used. Conveniently the enzyme may be produced using an induction agent, which may be the same as the intended substrate. An alternative protein expression system is a cell-free, in vitro expression system, in which a nucleotide sequence encoding the specific binding molecule is transcribed into mRNA, and the mRNA translated into a protein, in vitro. Cell-free expression system kits are widely available, and can be purchased from e.g., Thermo Fisher Scientific (USA).

As an alternative to production of the enzyme of the invention in a protein expression system, it may be chemically synthesised in a non-biological system, e.g., liquid-phase synthesis or solid-phase synthesis may be used.

The enzyme of the invention may, if desired, be provided in an isolated (i.e., purified) form. “Isolated”, as used herein, means that the enzyme is the primary component (i.e., majority component) of any solution or suchlike in which it is provided. In particular, if the enzyme is initially produced in a mixture or mixed solution, isolation of the enzyme means that it has been separated or purified therefrom. The enzyme may be present in the solution or composition at a purity of at least 60, 70, 80, 90, 95 or 99% w/w when assessed relative to the presence of other components, particularly other polypeptide components, in the solution or composition. A solution of the enzyme may be analysed by quantitative proteomics to identify the extent of purification of the enzyme of the invention, e.g., to assess if it is the predominant component. For instance, 2D gel electrophoresis and/or mass spectrometry may be used. Alternatively, the extent of purification may more simply be assessed by e.g., SDS-PAGE followed by Coomassie staining to check for contaminants/impurities.

The enzyme of the present invention may be isolated or purified using any technique known in the art. For instance, the enzyme may be produced with an affinity tag such as a polyhistidine tag (His tag), a strep tag, a FLAG tag, an HA tag or suchlike, to enable isolation or purification of the molecule by affinity chromatography using an appropriate binding partner, e.g., a molecule carrying a polyhistidine tag may be purified using Ni²⁺ ions. Alternatively, the enzyme may be isolated or purified by e.g., size-exclusion chromatography or ion-exchange chromatography.

Alternatively, the enzyme of the present invention may be provided in a non-purified form. For example it may be generated in a microorganism and the enzyme may be provided in a crude extract or lysate of that microorganism. Thus, in a preferred embodiment the enzyme is provided in the context of a microbial lysate. The enzyme may also be provided in an enriched extract in which the enzyme's concentration is increased relative to the concentration of other components in the lysate or extract. Once the enzyme is present at a concentration such that it is the most abundant polypeptide it may be considered isolated or purified as discussed above.

The enzyme (in purified or non-purified form) may be provided in a mixture with other components, e.g., components required for its function (as discussed further below), or other enzymes, such as other CYP450 enzymes. The enzyme may be provided in a solution or in a composition, e.g., with a suitable solution to maintain viability. The enzyme may also be provided in lyophilised form or immobilised or tethered to other macromolecules or support materials such as alginate beads, iron affinity beads, nickel columns and electrochemical electrodes.

A second aspect of the invention provides a nucleic acid molecule comprising a nucleotide sequence encoding the cytochrome P450 enzyme of the invention. A nucleotide sequence which encodes the enzyme of SEQ ID NO: 2 is set forth in SEQ ID NO: 117. It will be appreciated by those of ordinary skill in the art, however, that, as a result of the degeneracy of the genetic code, there are many nucleotide sequences that may encode any given amino acid sequence (such as that of SEQ ID NO: 2 or 3).

The nucleic acid molecule of the invention may be an isolated nucleic acid molecule and may further include DNA or RNA or chemical derivatives of DNA or RNA. The term “nucleic acid molecule” specifically includes single and double stranded forms of DNA and RNA. Methods for isolating or synthesising nucleic acid molecules are well known in the art.

The invention further provides a construct comprising the nucleic acid molecule of the invention. The construct is conveniently a recombinant construct comprising the nucleic acid molecule of the invention. In the construct, the nucleic acid molecule of the invention may be flanked by restriction sites (i.e., nucleotide sequences recognised by one or more restriction enzymes) to enable easy cloning of the nucleic acid molecule of the invention. In the construct of the invention the nucleotide sequence encoding the enzyme of the invention may conveniently be operably linked within said construct to an expression control sequence. The expression control sequence may be the natural sequence which controls expression of P450_{SeuC10} in S. eurythermus NRRL 2539, or a heterologous expression control sequence, i.e., an expression control sequence which does not control expression of P450_{SeuC10} in S. eurythermus NRRL 2539. Such an expression control sequence is typically a promoter, though the nucleotide sequence encoding the enzyme may alternatively or additionally be operably linked to other expression control sequences such as a terminator sequence, an operator sequence, an enhancer sequence or suchlike. Accordingly, the construct may comprise a promoter operably linked to the nucleic acid sequence encoding the enzyme of the invention. The promoter may be constitutive or inducible.

The term “operatively linked” (or “operably linked”) refers to the association of two or more nucleic acid molecules on a single nucleic acid fragment so that the function of one is affected by the other. For example, a promoter is operatively linked to a coding sequence when it is capable of affecting the expression of that coding sequence (i.e., the coding sequence is under the transcriptional control of the promoter). Coding sequences may be operatively linked to regulatory sequences in sense or antisense orientation.

The term “expression control sequence” refers to nucleotide sequences located upstream (5′ non-coding sequences), within, or downstream (3′ non-coding sequences) of a coding sequence, and which influence transcription, RNA processing or stability, or translation of the associated coding sequence. Expression control sequences may include promoters, operators, enhancers, translation leader sequences, a TATA box, a B recognition element and suchlike. As used herein, the term “promoter” refers to a nucleotide sequence capable of controlling the expression of a coding sequence or RNA. In general, a coding sequence is located 3′ to a promoter sequence. Promoters may be derived in their entirety from a native gene, or be composed of different elements derived from different promoters found in nature, or even comprise synthetic nucleotide segments. It is further recognised that since in most cases the exact boundaries of regulatory sequences have not been completely defined, nucleic acid fragments of different lengths may have identical regulatory activity.

Methods for preparing a nucleic acid molecule or construct of the invention are well known in the art, e.g., conventional polymerase chain reaction (PCR) cloning techniques can be used to construct the nucleic acid molecule of the invention which may be inserted into suitable constructs (e.g., containing an expression control sequence) using known methods.

The invention further provides a vector comprising a nucleic acid molecule or construct of the invention. The term “vector” as used herein refers to a vehicle into which the nucleic acid molecule or construct of the invention may be introduced (e.g., be covalently inserted) from which the enzyme or mRNA encoding it may be expressed and/or the nucleic acid molecule/construct of the invention may be cloned. The vector may accordingly be a cloning vector or an expression vector.

The nucleic acid molecule or construct of the invention may be inserted into a vector using any suitable methods known in the art, for example, without limitation, the vector and nucleic acid molecule may be digested using appropriate restriction enzymes and then may be ligated with the nucleic acid molecule having matching sticky ends, or as appropriate the digested nucleic acid molecule may be ligated into the digested vector using blunt-ended cloning.

The vector is generally a prokaryotic, specifically bacterial, vector. The nucleic acid molecule or construct of the invention may be produced in or introduced into a general-purpose cloning vector, particularly a bacterial cloning vector, e.g., an E. coli cloning vector. Examples of such vectors include pUC19, pBR322, pBluescript vectors (Stratagene Inc.) and pCR TOPO® from Invitrogen Inc., e.g., pCR2.1-TOPO.

The nucleic acid molecule or construct of the invention may be sub-cloned into an expression vector for expression of the enzyme of the invention. Expression vectors can contain a variety of expression control sequences. In addition to control sequences that govern transcription and translation, vectors may contain additional nucleic acid sequences that serve other functions, including for example vector replication, selectable markers etc. Plasmids are preferred vectors according to the invention.

The vector of the invention may further comprise a nucleotide sequence encoding a ferredoxin for use with the enzyme of the invention, as required for the enzyme's cytochrome P450 activity. In a particular embodiment, the vector may comprise a nucleotide sequence encoding the ferredoxin of SEQ ID NO: 4 (SeuF08, the native partner ferredoxin for P450_{SeuC10}). The native SeuF08 encoding sequence is set forth in SEQ ID NO: 118. In a particular embodiment, the vector comprises the nucleotide sequence of SEQ ID NO: 118, or a nucleotide sequence degenerate with SEQ ID NO: 118.

When the vector of the invention comprises both a nucleic acid molecule of the invention and a nucleotide sequence encoding a ferredoxin, the two genes may be encoded polycistronically, i.e., within an operon such that expression of both genes is controlled by the same promoter. Alternatively, the two genes may be encoded with separate promoters.

Alternatively or additionally, the vector of the invention may further comprise a nucleotide sequence encoding a ferredoxin reductase (e.g., a ferredoxin-NADP⁺-reductase) for use with the enzyme of the invention. Preferably the vector comprises nucleotide sequences (i.e., genes) encoding the enzyme of the invention, a ferredoxin and a ferredoxin reductase. The ferredoxin reductase may be encoded as part of an operon with the enzyme of the invention. In a particular embodiment the enzyme of the invention, ferredoxin and ferredoxin reductase are encoded in a single operon. The genes may be encoded in any order within such an operon.

In a particular embodiment the vector encodes the ferredoxin reductase Scf15A. Scf15A has the amino acid sequence set forth in SEQ ID NO: 119. In a particular embodiment, the vector may comprise a nucleotide sequence encoding the ferredoxin reductase of SEQ ID NO: 119. The native Scf15A coding sequence (Streptomyces coelicolor A3(2)) is set forth in SEQ ID NO: 120. In a particular embodiment, the vector comprises the nucleotide sequence of SEQ ID NO: 120, or a nucleotide sequence degenerate with SEQ ID NO: 120.

The invention further provides a microorganism comprising the nucleic acid molecule of the invention, the recombinant construct of the invention or the vector of the invention (which is heterologous to the microorganism); or a lysate of such a microorganism. The microorganism is generally a prokaryote, particularly a bacterium. The bacterium may be a Gram-positive or Gram-negative species or strain, generally a non-pathogenic bacterium. In a preferred embodiment, the bacterium is Escherichia coli. The microorganism may be a cloning host or an expression host. Suitable bacterial expression strains are known, e.g., E. coli expression strains, such as E. coli (DE3) strains. The host may also contain nucleic acid encoding one or more cofactor enzymes, e.g., ferredoxin and ferredoxin reductase. This can be achieved by polycistronic plasmid use or via fusion, either via linkers or directly into a single protein product.

A lysate of the invention (i.e., a lysate of a microorganism of the invention) comprises the enzyme of the invention. Thus the lysate is a lysate of a microorganism that expresses the enzyme (particularly of a bacterium that expresses the enzyme). Such a lysate may be obtained using standard methods of microorganism cell lysis. For instance, the microorganism may be mechanically lysed (e.g., by French press), acoustically lysed (e.g., by sonication), chemically lysed using an appropriate lysis buffer/reagent (e.g., BugBuster, Sigma Aldrich, USA) or lysed by freeze-thaw cycle(s). Partial lysis or permeabilisation may be performed to generate the lysate, e.g., to produce a cell paste. By way of example, the lysate may be a cell (e.g., E. coli) paste, e.g., that has been partially lysed or permeabilised by a freeze-thaw process. The lysate may be a raw lysate, i.e., subjected to no additional treatment following lysis. Alternatively, the lysate may be processed, e.g., the insoluble fraction may be removed (e.g., by centrifugation) such that only the soluble fraction of the lysate is provided. The resulting soluble fraction may be frozen for later use as described below, or in a preferred embodiment the frozen soluble fraction is lyophilised and preferably the container vessels, e.g., vials containing the resulting lyophilisate, are sealed under vacuum. A lysate of the invention thus may be a raw (unprocessed) lysate or a lysate which has been enriched for the enzyme of the invention relative to the raw lysate. This may also be referred to as an extract.

In a further aspect, the invention provides a kit comprising:
(i) an enzyme of the invention; and/or
(ii) a microorganism that expresses an enzyme of the invention, preferably a microorganism of the invention, or a lysate thereof; and/or
(iii) a nucleic acid molecule of the invention, a recombinant construct of the invention or a vector of the invention.

The kit components, such as the cytochrome P450 enzyme, microorganism or lysate (which may be fresh, frozen and/or lyophilised), or nucleic acid molecule, recombinant construct or vector may be lyophilised and/or vacuum sealed.

In a preferred embodiment, the kit further comprises electron donating agents (i.e., a reducing agent). This is particularly advantageous when the kit comprises the enzyme of the invention. The kit preferably comprises as the electron donating agents a ferredoxin reductase and a ferredoxin, preferably with cofactors NADH or NADPH or cofactor regeneration systems such as NAD+ or NADP+, glucose or glucose-6-phosphate, and glucose-dehydrogenase or glucose-6-phosphate dehydrogenase. However, any suitable electron donating agents may be used.

Optionally, the kit may further comprise a buffer, either separately or contained with the other components. This is particularly advantageous when the kit comprises the enzyme of the invention.

Preferably, the kit may further comprise one or more other CYP450 enzymes. This is particularly advantageous when the kit comprises the enzyme of the invention. When the kit comprises a microorganism expressing the enzyme of the invention, the microorganism may further express one or more additional CYP450 enzymes, or the kit may comprise one or more additional microorganisms, or their lysates, each of which expresses a further CYP450 enzyme (i.e., a CYP450 enzyme other than that of the invention). When the kit comprises a nucleic acid molecule, recombinant construct or vector encoding the enzyme of the invention, the nucleic acid molecule, recombinant construct or vector may further encode one or more additional CYP450 enzymes, or the kit may further comprise one or more additional nucleic acid molecules, recombinant constructs or vectors, each of which encodes a further CYP450 enzyme (i.e., a CYP450 enzyme other than that of the invention).

Preferably, the cytochrome P450 enzyme or microorganism or its lysate is lyophilised or immobilised or tethered to other macromolecules or support materials such as alginate beads, iron affinity beads, nickel columns and electrochemical electrodes.

In a further aspect the invention provides a method of producing an oxidised organic compound, comprising contacting the organic compound with the enzyme of the invention, in order to oxidise said organic compound. In a related aspect the invention provides the use of the enzyme of the invention to oxidise an organic compound.

When the enzyme of the invention is combined with suitable reductase components, it is able to oxidise organic compounds. A variety of different compounds can be oxidised (e.g., hydroxylated, carboxylated, dealkylated, preferably demethylated, epoxidated, etc.) using the claimed cytochrome P450 enzyme. In a preferred embodiment, the organic compound to be oxidised will have a rate of conversion to the resulting derivative of at least 3%, more preferably at least 5%, more preferably at least 10%, more preferably at least 25%, more preferably at least 50%, even more preferably at least 70% and most preferably a rate of conversion to the resulting derivative of 100% using the same conditions described in Example 5 herein.

In an embodiment the oxidation performed by the enzyme of the invention is hydroxylation, epoxidation, carboxylation or dealkylation, preferably demethylation. Preferably the enzyme is used to catalyse the oxidation of an alkyl or aryl group. In a particularly preferred aspect the enzyme catalyses oxidation (particularly hydroxylation) of an aromatic ring on an organic compound. The compound to be oxidised by the cytochrome P450 enzyme may have an optionally substituted or unsubstituted linear or branched alkyl group, such as methyl, isopropyl or tert-butyl, which is hydroxylated; or an aromatic group, such as an optionally substituted aryl or heteroaryl, which is hydroxylated; or an olefinic group, or substituted aryl or heteroaryl, which is epoxidated; or an alkyl-heteroatom, which is dealkylated.

Preferably, the compound to be oxidised is of formula I:

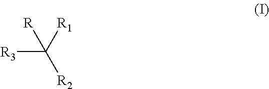

where R represents the rest of the compound, and where R¹, R² and R³ are independently selected from H or C₁₋₁₂ alkyl or C₆₋₁₀ aryl, or wherein any two of R¹, R² and R³ may be joined to form an optionally substituted cycloalkyl or heterocycloalkyl or R¹, R² and R³ may be joined together with their bridging carbon to form an olefin, aryl or heteroaryl.

Preferably R is an optionally substituted alkyl; an optionally substituted olefin, an optionally substituted aryl, optionally substituted heteroaryl or optionally substituted heterocycloalkyl.

As used herein “alkyl” means a C₁-Cₙ alkyl group, which can be linear or branched or cyclic. Examples include propyl and butyl, pentyl, hexyl, cyclopentyl and cyclohexyl. Preferably, it is a C₁-C₁₀ alkyl moiety, e.g., a C₃-C₁₀ alkyl moiety. More preferably it is a C₅-C₆ alkyl moiety. Preferably the alkyl is an optionally substituted cyclohexyl. For the avoidance of doubt, the term cycloalkyl is a cyclic alkyl group.

As used herein “aryl” means an optionally substituted monocyclic, bicyclic or tricyclic aromatic radical, such as phenyl, biphenyl, naphthyl and anthracenyl. Preferably the aryl is an optionally substituted Ce aryl.

As used herein “heteroaryl” means an optionally substituted monocyclic, bicyclic or tricyclic aromatic radical containing at least one and up to four heteroatoms selected from oxygen, nitrogen and sulfur, such as furanyl, pyrrolyl, thiazolyl, isothiazolyl, tetrazolyl, imidazolyl, oxazolyl, isoxazolyl, thienyl, pyrazolyl, pyridinyl, pyrazinyl, pyrimidinyl, indolyl, azaindolyl, isoindolyl, quinolyl, isoquinolyl, triazolyl, thiadiazolyl, oxadiazolyl.

As used herein heterocycloalkyl means an optionally substituted cycloalkyl wherein one to four carbon atoms have been substituted with a heteroatom. Preferably, the heteroatoms are selected from nitrogen, oxygen, sulphur or phosphorous.

As used herein the term “optionally substituted” means an H has been removed from a compound and replaced with an organic fragment such as those comprising a combination of any of carbon, a halogen, hydrogen, nitrogen, oxygen and sulphur. Preferably the compound of formula I has a molecular weight of from 50 to 1000, such as from 100 to 700, more preferably from 200 to 500.

Preferably, R¹, R² and R³ are independently selected from H, C₁₋₆ alkyl or C₆₋₁₀ aryl, preferably with the proviso that either one or more of R¹, R² and R³ is H. Most preferably, R¹, R² and R³ are independently selected from H, methyl, ethyl, propyl, butyl, t-butyl, pentyl and hexyl preferably with the proviso that either one or more of R¹, R² and R³ is H.

In a particularly preferred embodiment, the cytochrome P450 enzyme is reacted with a compound such as carbamazepine (compound (i) below), bosentan (compound (ii) below), diclofenac (compound (iii) below), meloxicam (compound (iv) below), tivantinib (compound (v) below), or ambroxide (compound (vi) below). The enzyme may also be reacted with any of the other compounds described in the Examples, e.g., clarithromycin or sclareol.

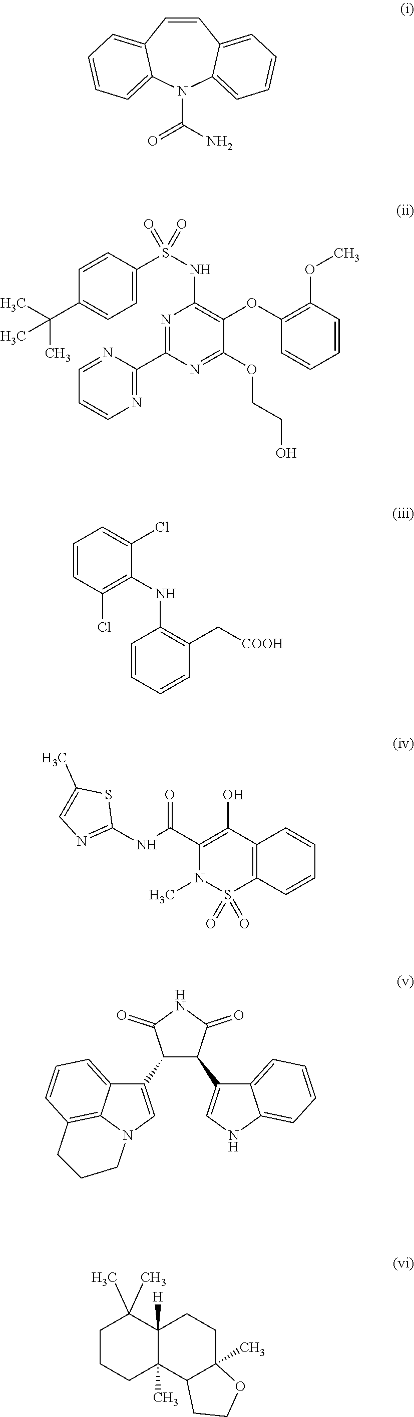 [SMILES: NC(=O)N1c2ccccc2C=Cc2ccccc21 ; COc1ccccc1Oc1c(NS(=O)(=O)c2ccc(C(C)(C)C)cc2)nc(-c2ncccn2)nc1OCCO ; O=C(O)Cc1ccccc1Nc1c(Cl)cccc1Cl ; Cc1cnc(NC(=O)C2=C(O)c3ccccc3S(=O)(=O)N2C)s1 ; O=C1NC(=O)[C@@H](c2cn3c4c(cccc24)CCC3)[C@@H]1c1c[nH]c2ccccc12 ; CC1(C)CCC[C@]2(C)C3CCO[C@]3(C)CC[C@@H]12]

In alternatively described preferred aspects, the organic compound to be oxidised may be selected from a terpenoid (e.g., an aliphatic terpenoid such as ambroxide) and an aromatic compound (such as carbamazepine). A “terpenoid” is a compound derived from isoprene, in line with standard nomenclature in the art. Aromatic compounds include one or more aromatic rings. In a particular embodiment, the organic compound to be oxidised may be a sesquiterpene (i.e., a terpene comprising three isoprene units), particularly a cyclic sesquiterpene, such as ambroxide or sclareol. In an alternative the organic compound to be oxidised may be a macrolide antibiotic, such as azithromycin, clarithromycin, erythromycin or roxithromycin. In a further alternative the enzyme may be used to generate a macrolide antibiotic by the method described herein using an enzyme of the invention.

The enzyme of the invention may be used in combination with reductase components, which activate the cytochrome P450. In a preferred embodiment, ferredoxin and ferredoxin reductase components are used. In particular, the enzyme of the invention may be used in combination with the ferredoxin SeuF08 of SEQ ID NO: 4, or a variant thereof having at least 80, 85, 90 or 95% sequence identity to SEQ ID NO: 4 and which retains the ferredoxin activity of SeuF08. The enzyme of the invention may be used in combination with the ferredoxin reductase Scf15A of SEQ ID NO: 119, or a variant thereof having at least 80, 85, 90 or 95% sequence identity to SEQ ID NO: 119 and which retains the ferredoxin activity of Scf15A. Preferably the enzyme of the invention is used in combination with SeuF08 and Scf15A, or variants thereof.

Any components which activate the cytochrome P450 may also or alternatively be used, including those fused directly or by peptide linkage, or chemical-based oxygen providing surrogates such as peroxide, iodane or chemicals of similar resulting properties.

The enzyme of the invention may be used in purified (i.e., isolated) form, part-purified (i.e., part-isolated) form, in a cell lysate or extract as discussed above or within a recombinant host cell (i.e., a recombinant microorganism). In the instance that the enzyme is used in a cell lysate or recombinant host cell the cell lysate or host cell is generally a cell lysate or microorganism of the invention.

When the enzyme is used in purified or part-purified form, or in the context of a cell lysate, the compound to be oxidised is contacted with the (part-)purified enzyme or lysate or extract, and optionally any additional components as described above.

Alternatively, as noted above the enzyme may be used when it is within a host cell, i.e., a host cell may be used for biotransformation of the substrate compound. In this case the cells may be dosed with the organic compound to be oxidised.

The enzyme, lysate or host strain is contacted directly with the substrate, preferably in an aqueous medium, either mono or biphasic. Reaction conditions, including choice of pH and temperature will be evident to the skilled person, based on conventional techniques. For example, the reaction may be performed at a pH value in the range of from 5 to 11, more preferably 6.5 to 9.0, most preferably around 8 may be used. To achieve this pH, a selected microbial growth medium or phosphate buffer solution may be used which has the above-mentioned pH. The reaction temperature is preferably within the range from 20° C. to 45° C., more preferably from 25° C. to 30° C. The concentration of the substrate in the reaction medium is preferably within the range from 0.01 to 5.0% by weight. The time allowed for the reaction is normally from 1 minute to 5 days, more usually from 1 day to 5 days, although this may vary, depending upon the concentration of substrate in the reaction mixture, the reaction temperature, and other factors. The extracted enzyme material can either be used directly after extraction, or after storage in frozen solution. In a particularly preferred embodiment, the extracted enzyme material can be dried, preferably by lyophilisation, with or without vessel closure under vacuum, for later use with or without the addition of other components required for reaction, such as other enzyme cofactor components.

The method may optionally comprise a subsequent step wherein the cells are harvested and optionally the oxidised compound may be enriched, purified (or isolated). The cells are harvested or collected by any suitable means (e.g., by centrifugation). If the oxidised compound is secreted by the cells, such that it is present in the cell supernatant, the oxidised compound is collected/harvested and/or isolated from the supernatant. Such isolation may be performed using standard methods in the art, as discussed below. If the oxidised compound is present in the cells, the cells may be lysed, e.g., using methods as described above, and the oxidised compound isolated from the lysate. Such isolation may be performed by standard methods in the art, as discussed below.

After completion of the oxidation reaction, the resulting oxidised compound can be isolated (or purified) using conventional procedures, including, for instance, filtration, solvent extraction, chromatography, crystallization, and other isolation procedures. Such procedures will be selected having due regard to the identity of the product. Before, during or after the isolation, the product may or may not be derivatised, as desired. Isolation and purification are referred to herein, in some cases interchangeably. Isolation may be considered a form of purification or the first step of purification, e.g., separation of the oxidised compound from the bulk reaction. Further purification steps may then be conducted to achieve improved purity. Thus reference to isolation herein may be considered a first purification step. Purification may comprise only a first step of isolation, but may also include additional steps to achieve higher levels of purity. Preferably purity levels of at least 80, 85, 90 or 95% w/w (dry weight) are achieved for the oxidised compound. Enriched preparations may have lower levels of the oxidised product but have higher levels of that product than the levels present in the reaction mix immediately after the oxidation reaction.

The starting materials used as substrates for the enzyme may be derived from synthetic routes or may be naturally occurring, e.g., via natural biomass such as plant material, or produced by fermentation, or by mixed routes thereof. Enzyme reactions can also be performed using pure or non-purified materials, and may be used to aid later purifications of reacted or unreacted components. Of the substrate compounds used as starting materials, free bases, alkali metal salts, e.g., sodium or potassium salts, or acid salts of organic or inorganic nature such as tosylate or hydrochlorides, are particularly suitable for use.

After completion of the conversion reaction, the desired compound can be obtained from the reaction system, harvested/collected, isolated, enriched and/or purified by conventional means if required, or onward used directly in unpurified form. For example, the reaction product may be centrifuged or filtered and the supernatant or filtrate extracted with a hydrophobic resin, ion-exchange resin or water-immiscible organic solvent such as ethyl acetate. After evaporation of the solvent of the extract, the remaining crude material, for example the remaining crude oxidised compound, may be purified by subjecting it to column chromatography using silica gel or alumina or reverse-phase stationary phase, and by eluting with a suitable eluent. If the starting material is a mixture, then the product can be isolated as a mixture of oxidised compounds which if desired can be separated using chromatography or other suitable techniques. The oxidised compound may be further processed or packaged for commercial use.

In general, the resulting oxidised compound may have improved pharmaceutical or agrochemical properties, such as bioactivity potency, improved solubility characteristics, reduced off-target interactions, or simply be of further utility, such as for onward synthesis, or be useful for an analytical standard.

The enzyme of the invention may be used to oxidise any suitable organic substrate. In a particular embodiment, the enzyme of the invention is used to hydroxylate an organic substrate. The oxidisation, e.g., hydroxylation, may preferably be performed on an aromatic ring of the organic substrate, i.e., the enzyme preferably catalyses oxidation, e.g., hydroxylation, of an aromatic ring on the organic substrate.

Whilst the enzymes of the invention may be used to oxidise any suitable organic substrate, particularly those described hereinbefore, as described herein, the CYP450 enzymes of the invention have been found to display particular and novel activity towards aliphatic terpenoid and aromatic compound substrates. Thus in an embodiment of the invention the enzyme of the invention is used to oxidise (e.g., hydroxylate) an aromatic compound substrate and/or a terpenoid substrate, preferably to oxidise an aliphatic terpenoid substrate, especially preferably a sesquiterpene substrate.

In particular, CYP450 enzymes of the invention in which the amino acid at the position corresponding to position 109 of SEQ ID NO: 3 is substituted from the native threonine residue have been found to display modified regioselectivity towards the aliphatic terpenoid compound ambroxide. This is particularly the case for CYP450 enzymes of the invention comprising a phenylalanine residue at the position corresponding to position 109 of SEQ ID NO: 3, such as the enzyme of SEQ ID NO: 54. Thus in a particular embodiment of the invention, an enzyme of the invention comprising an amino acid other than threonine at the position corresponding to position 109 of SEQ ID NO: 3 is used to oxidise (e.g., hydroxylate) a terpenoid compound, preferably an aliphatic terpenoid. The enzyme of the invention used to oxidise the terpenoid compound preferably has a phenylalanine, alanine or valine residue at the position corresponding to position 109 of SEQ ID NO: 3, most preferably phenylalanine.

CYP450 enzymes of the invention in which the amino acid at the position corresponding to position 198 of SEQ ID NO: 3 is substituted from the native leucine residue have been found to display modified regioselectivity towards the aromatic compound carbamazepine. This is particularly the case for CYP450 enzymes of the invention comprising a phenylalanine, alanine or tryptophan residue at the position corresponding to position 198 of SEQ ID NO: 3, such as the enzymes of SEQ ID NOs: 22, 30 and 44. Thus in a particular embodiment of the invention, an enzyme of the invention comprising an amino acid other than leucine at the position corresponding to position 198 of SEQ ID NO: 3 is used to oxidise (e.g., hydroxylate) an aromatic compound. The enzyme of the invention used to oxidise the aromatic compound preferably has a phenylalanine, alanine or tryptophan residue at the position corresponding to position 198 of SEQ ID NO: 3.

When the enzymes of this invention are reacted with substrate compound at pH 8.0 for 5 minutes with (a) ferredoxin, (b) ferredoxin-NADP⁺-reductase, (c) NADPH regeneration system, and (d) dissolved oxygen, the temperature of reaction ranges at least from 4° C. to 60° C. The optimum pH for each cytochrome ranges from 6.5 to 8.0. Each cytochrome is stable when kept for 24 hours at 4° C. in the pH range between 6.0 and 9.0. Stored lyophilised enzyme is stable at 20-27° C. for 10 days compared to a control stored at <−18° C.

The use of ferredoxin, ferredoxin-NADP⁺-reductase, oxygen and NADPH is not essential. Any components which can activate the cytochrome P450 may be adopted.

Measurement of the activity of a cytochrome P450 enzyme is normally effected in one of two ways:

### (i) Measurement on Cytochrome P450:

Measurement is performed according to the method of Omura and Sato et al. (J Biol Chem, 239. 1964, 2370). That is to say, cytochrome P450 is analyzed quantitatively using the following formula, based on the difference in the absorbance of the reduced CO versus the reduced difference spectrum at 450 nm and 490 nm.

$$Cytochrome P 450 ( mM ) = \frac{Abs ( 450 nm ) - Abs ( 490 nm )}{91 ( mM {cm}^{- 1} ) \times l ( cm )}$$

(ii) Measurement of Rate of Formation of Oxidised Substrate Compound from Substrate Compound:

### The Following Cocktail of Components is Employed:

| | |
|---|---|
| Potassium phosphate buffer pH 8.0 | 100 mM |
| MgCl₂ | 5 mM |

Enzyme solution containing expressed ferredoxin, ferredoxin reductase and P450 Native concentration when pellet extracted at a rate of 0.30 g cell wet weight per ml extraction buffer

| | | |
|---|---|---|
| NADP⁺ | 1 | mM |
| Glucose-6-phosphate | 5 | mM |
| Glucose-6-phosphate dehydrogenase | 1 | UN/ml |
| Substrate compound | 0.1 | mg/ml |

| | |
|---|---|
| Total volume | e.g., 0.1-0.5 ml |

To measure enzyme activity the components listed above are mixed, the solution is shaken at 27° C. for 16-20 hours, and then e.g., 100-500 μl of ACN is added and the reaction stopped. The amount of oxidised substrate formed by the enzyme system is determined with HPLC or UPLC. The reaction may be used on a preparative scale by increasing the volume as appropriate.

Measurement of CYP450 activity may also be performed as demonstrated in the Examples below.

In a further aspect the invention provides a method of producing a cytochrome P450 enzyme of the invention, the method comprising introducing a nucleic acid molecule, recombinant construct or vector of the invention into a microorganism, and expressing the cytochrome P450 enzyme in the microorganism, and optionally purifying (or isolating) the cytochrome P450 enzyme.

Techniques for performing the method are well known in the art. The nucleic acid molecule, recombinant construct or vector may be generated using standard techniques, as described above. The microorganism into which the nucleic acid molecule, construct or vector is introduced is preferably as described above in the context of the microorganism of the invention. That is to say, the microorganism is preferably a bacterium, e.g., E. coli. The enzyme is expressed in the microorganism using standard techniques in the art (e.g., as demonstrated in the Examples below).

To obtain active enzyme the microorganism may be lysed, to provide a lysate comprising the enzyme. Lysis may be performed using standard methods in the art, e.g., French press. The enzyme may then be purified (or isolated), if desired. Purification may be performed using standard methods in the art. For example, the enzyme may be expressed with an affinity tag (e.g., a His tag or a Strep tag) and then purified by affinity chromatography, as described above.

The methods of the present invention are demonstrated in the examples below. These examples are provided as an illustration only and should not be construed as limiting on the present invention.

### EXAMPLES

### Example 1: Cloning of P450_{SeuC10} from Streptomyces eurythermus NRRL 2539

Extraction of genomic DNA from Streptomyces eurythermus NRRL 2539 Genomic DNA (gDNA) was isolated from a cell pellet of Streptomyces eurythermus NRRL 2539. Culture medium contained 4 g/L yeast extract; 10 g/L malt extract; 4 g/L glucose and was adjusted to pH 7.0. Two Erlenmeyer flasks of 250 ml volume, each of which contained 50 ml of the medium, were sterilized at 115° C. for 20 minutes. Streptomyces eurythermus NRRL 2539 was recovered from cryovial stocks stored in liquid nitrogen and inoculated into the two flasks containing 50 ml of the above growth medium. After 2 days of growth at 27° C. and 200 rpm, 50 mls of culture were transferred to 50 ml centrifuge tubes and centrifuged to collect the pelleted cells. The pellet was washed once with an isotonic buffer to remove residual medium components before freezing the pellet at −80° C. for later extraction of genomic DNA as described below. The cell pellet was defrosted and resuspended in 7.5 ml TE buffer (10 mM Tris-HCl PH 7.5, 1 mM Na₂EDTA). 75 μl of 20 mg/ml lysozyme solution was added and the solution was incubated at 37° C. for 1 hour, followed by addition of 750 μl of 10% (w/v) SDS and mixing by inverting. After addition of 20 μl of 20 mg/ml pronase and incubation at 37° C. for 1.5 hours, the solution was supplemented with 16 μl of 10 mg/ml RNase solution, followed by another incubation step at 37° C. for 1 hour and 50° C. for 1 hour. 900 μl of 0.5 M NaCl solution was added before the solution was extracted twice with an equal volume of phenol:chloroform:isoamyl-alcohol (25:24:1; Sigma-Aldrich). The aqueous layers were collected and gDNA was precipitated with 1 volume of isopropanol and centrifugation (10,000×g, 30 min, 20° C.). The gDNA pellet was washed once with 100% ethanol and twice with 70% ethanol (˜30 ml each wash step). The gDNA pellet was air-dried and resuspended in 5 ml TE buffer. Concentration and purity of the gDNA was measured using a NanoDrop instrument (Thermo Scientific) and gDNA integrity was assessed by agarose gel electrophoresis.

### PCR Reactions

The P450_{SeuC10} and ferredoxin_{SeuF08} gene operon (SEQ ID NO: 1) was cloned from Streptomyces eurythermus NRRL 2539 in a total reaction volume of 50 μl using primers SeuC10-SeuF08_f (5′-primer sequence-3′: ATTTTGTTTAACTTTAAGAAGGAGATATACATATGAAGATCGGCACGACGCACCTC) (SEQ ID NO: 59) and SeuC10-SeuF08_r (5′-primer sequence-3′: CTACCCGCAGAGGGGGGGGCATAAGCTTCCTATTAGGCGGAGCGCTCCCGTACGGTG ATG) (SEQ ID NO: 60). PCR reactions contained 10 μl of 5× GC Green buffer (Thermo Scientific), 2.5 μl of DMSO (Sigma), 10 μl of 5 M betaine (Sigma), 1 μl of formamide (Sigma), 1 μl of 10 mM dNTPs (Thermo Scientific), 1 unit of HotStart II Phusion® High-Fidelity DNA Polymerase (Thermo Scientific), ˜90 ng of genomic DNA and 0.5 UM of each forward and reverse primer. The reaction mix was topped up to a total volume of 50 μl with MilliQ®-H₂O. PCR reactions were performed on an Eppendorf Mastercycler ep Gradient system with the following cycling conditions: 98° C. for 2 minutes, 35 cycles (98° C. for 45 seconds, 72° C. for 30 seconds, and 72° C. for 3 minutes), 72° C. for 15 minutes. The PCR reaction was analysed by agarose gel electrophoresis and products were extracted from the agarose gel using the Qiagen QIAquick 96 PCR Purification Kit. The concentration of the expected 1558 bp amplicon was measured using the Biochrome Genequant 1300 instrument and on the Molecular Devices Spectramax 384 plus plate reader.

Construction of pHD05 Vector

The pHD05 vector is a derivative of pHD02 (See WO 2018/091885) containing the cer sequence. The cer sequence was amplified from pKS450 plasmid (Summers and Sherratt., EMBO J. 1988; 7(3):851-858.) by PCR using the primers ser_f (5′-primer sequence-3′: GGGTCCTCAACGACAGGAGCACGATCATGCCGGAAATACAGGAACGCACGCTG) (SEQ ID NO: 61) and ser_r (5′-primer sequence-3′: TTATCGCCGGCATGGCGGCCCCACGGGTGCCGGGGCACAACTCAATTTGCGGGTAC) (SEQ ID NO: 62). The expected 439 bp amplicon was extracted from agarose gel using the Thermofisher GeneJet Gel Extraction Kit and cloned into the FspAl site of pHD02 by Gibson assembly. The plasmids containing the cer sequence were analysed by PCR screening and the DNA sequence was confirmed by Sanger sequencing at LGC Genomics (Germany). The plasmid containing the cer sequence was designated as pHD05.

Cloning of the P450_{SeuC10} and Ferredoxin_{SeuF08} Gene Operon into pHD05 Plasmid

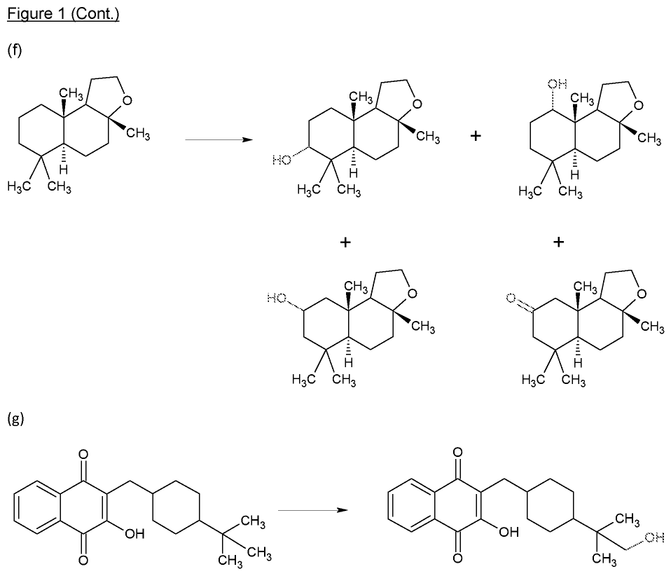

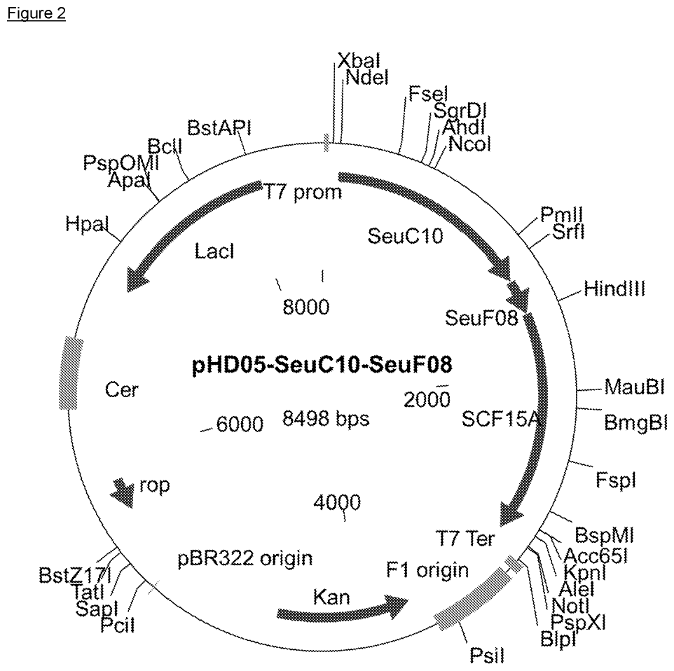

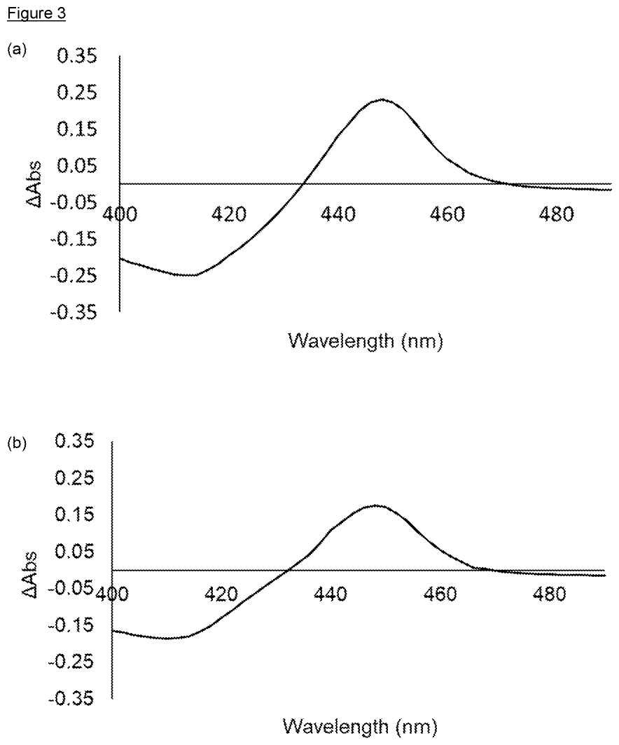

The purified P450_{SeuC10} and ferredoxin_{SeuF08} amplicon was assembled into the pHD05 vector digested with Ndel and Ecorl, so that the cytochrome P450 and ferredoxin gene operon was introduced into a polycistronic operon containing a ferredoxin reductase (scf15a). The vector was digested with restriction endonuclease (New England Biolabs). Restriction digestion was carried out for 16 hours at 37° C. in a total volume of 200 μl containing 20 μl of 10× CutSmart buffer®, 4 μl of each restriction endonuclease (40 units; New England Biolabs), and ˜10.4 μg of plasmid DNA. The reaction was stopped by inactivation of the restriction endonuclease at 65° C. for 20 min. The expected digested products were purified using the Thermo Scientific GeneJET Gel Extraction Kit. The purified digested vector and purified P450 amplicon were assembled together using Gibson assembly in a total volume of 20 μl containing ˜50 ng of digested vector and 1:3 (vector:insert) molar concentration of insert, 6.65% PEG 8000, 133 mM Tris-HCl (Fisher) pH 7.5, 13.3 mM MgCl₂ (Sigma), 13.3 mM DTT (Sigma), 0.266 mM dNTP (New England Biolabs), 1.33 mM NAD (New England Biolabs), 0.495 Unit of Phusion DNA polymerase (New England Biolabs), 79.5 Units of Taq DNA ligase (New England Biolabs) and 0.075 Units of T5 exonuclease (New England Biolabs). The reaction mixture was incubated at 50° C. for 1 hour and 1 μl (˜100 ng) was introduced into 25 μl of chemically competent cells E. coli DH5a (Invitrogen) by chemical transformation. The transformation mixture was incubated on ice for 30 minutes and heat-shocked at 42° C. for 30 seconds. 1 ml of Miller's Luria Broth (LB) was immediately added and the mixture was further incubated at 250 rpm at 37° C. The transformation mixture was then plated onto LB agar plates containing 50 μg/ml kanamycin and incubated at 37° C. for 16 hours. Clones were picked and cultivated in 5 ml LB containing the same antibiotic and recombinant plasmids were isolated from the cultures using the QIAGEN QIAprep 96 Plus Kit. DNA sequences of the P450, ferredoxin and ferredoxin reductase were analysed by PCR screening and DNA sequence were confirmed by Sanger sequencing at LGC genomics (Germany). The constructed plasmid was designated as pHD05-SeuC10-SeuF08 (FIG. 2).

### Example 2: Site-Directed Mutagenesis of P450_{SeuC10}

### PCR-Based Site Directed Mutagenesis

Site directed mutagenesis by PCR of positions R⁹⁰, R¹⁰¹, R¹⁰⁶, R¹⁰⁸, T109, L198, R²¹⁰ and L259 in P450_{SeuC10} was performed in which the amino acid codon was altered to encode a new amino acid. One new amino acid was chosen to modify position 90. Three new amino acids were chosen to modify position 101. Three new amino acids were chosen to modify position 106. One amino acid was chosen to modify position 108. Seven new amino acids were chosen to modify position 109. Eleven new amino acids were chosen to modify position 198. One new amino acid was chosen to modify position 210. Two new amino acids were chosen to modify position 259. Three double mutants were generated with a mutation at position 109 and a mutation at position 198 or 259. The sequences of primers used for amplification and mutagenesis are shown in Table 1 below. Two oligonucleotides were designed that are complementary to the sequences surrounding the triplet encoding the specific residue to mutate.

TABLE 1
Primers used for amplification and mutagenesis

| Mutant | Primer Sequences |
|---|---|
| Mutant 1 | CGGCCGCGACCGCCGTCTGGCCCTGCTC |
| (T109R) | (SEQ ID NO: 63) |
| | GAGCAGGGCCAGACGGCGGTCGCGGCCG |
| | (SEQ ID NO: 64) |
| Mutant 2 | GCCGCGACCGCGCGCTGGCCCTGCTC |
| (T109A) | (SEQ ID NO: 65) |
| | GAGCAGGGCCAGCGCGCGGTCGCGGC |
| | (SEQ ID NO: 66) |
| Mutant 3 | CACCACGGACGCCGCGGACCGGCTGGAG |
| (R210A) | (SEQ ID NO: 67) |
| | CTCCAGCCGGTCCGCGGCGTCCGTGGTG |
| | (SEQ ID NO: 68) |
| Mutant 4 | CGGCCGGCCGCGACGCGACGCTGGCCCTG |
| (R108A) | (SEQ ID NO: 69) |
| | CAGGGCCAGCGTCGCGTCGCGGCCGGCCG |
| | (SEQ ID NO: 70) |
| Mutant 5 | CTTCGCGGCCGGCGCGGACCGCACGCTG |
| (R106A) | (SEQ ID NO: 71) |
| | CAGCGTGCGGTCCGCGCCGGCCGCGAAG |
| | (SEQ ID NO: 72) |
| Mutant 6 | GTGCCCACCCCCGCGTTCGCGGCCGGCC |
| (R101A) | (SEQ ID NO: 73) |
| | GGCCGGCCGCGAACGCGGGGGTGGGCAC |
| | (SEQ ID NO: 74) |
| Mutant 7 | GTCCACCGACGCGCGCCGCGACGGCTTC |
| (R90A) | (SEQ ID NO: 75) |
| | GAAGCCGTCGCGGCGCGCGTCGGTGGAC |
| | (SEQ ID NO: 76) |
| Mutant 8 | GTCCCGCCGGCTGATTCGCGGCCCGAC |
| (L1981) | (SEQ ID NO: 77) |
| | GTCGGGCCGCGAATCAGCCGGCGGGAC |
| | (SEQ ID NO: 78) |
| Mutant 9 | GTCCCGCCGGCTGGCGCGCGGCCCGAC |
| (L198A) | (SEQ ID NO: 79) |
| | GTCGGGCCGCGCGCCAGCCGGCGGGAC |
| | (SEQ ID NO: 80) |
| Mutant 10 | GTCGCTCGCCGTCCTGCTGCTGGTCGCC |
| (1259L) | (SEQ ID NO: 81) |
| | GGCGACCAGCAGCAGGACGGCGAGCGAC |
| | (SEQ ID NO: 82) |
| Mutant 11 | GTCGCTCGCCGTCGCGCTGCTGGTCGCC |
| (1259A) | (SEQ ID NO: 83) |
| | GGCGACCAGCAGCGCGACGGCGAGCGAC |
| | (SEQ ID NO: 84) |
| Mutant 12 | GTCCCGCCGGCTGATGCGCGGCCCGAC |
| (L198M) | (SEQ ID NO: 85) |
| | GTCGGGCCGCGCATCAGCCGGCGGGAC |
| | (SEQ ID NO: 86) |
| Mutant 13 | GTCCCGCCGGCTGTTTCGCGGCCCGAC |
| (L198F) | (SEQ ID NO: 87) |
| | GTCGGGCCGCGAAACAGCCGGCGGGAC |
| | (SEQ ID NO: 88) |
| Mutant 14 | GTCCCGCCGGCTGGTGCGCGGCCCGAC |
| (L198V) | (SEQ ID NO: 89) |
| | GTCGGGCCGCGCACCAGCCGGCGGGAC |
| | (SEQ ID NO: 90) |
| Mutant 15 | GTCCCGCCGGCTGACCCGCGGCCCGAC |
| (L198T) | (SEQ ID NO: 91) |
| | GTCGGGCCGCGGGTCAGCCGGCGGGAC |
| | (SEQ ID NO: 92) |
| Mutant 16 | GTCCCGCCGGCTGAACCGCGGCCCGAC |
| (L198N) | (SEQ ID NO: 93) |
| | GTCGGGCCGCGGTTCAGCCGGCGGGAC |
| | (SEQ ID NO: 94) |
| Mutant 17 | GTCCCGCCGGCTGGATCGCGGCCCGAC |
| (L198D) | (SEQ ID NO: 95) |
| | GTCGGGCCGCGATCCAGCCGGCGGGAC |
| | (SEQ ID NO: 96) |
| Mutant 18 | GTCCCGCCGGCTGCATCGCGGCCCGAC |
| (L198H) | (SEQ ID NO: 97) |
| | GTCGGGCCGCGATGCAGCCGGCGGGAC |
| | (SEQ ID NO: 98) |
| Mutant 19 | GTCCCGCCGGCTGTATCGCGGCCCGAC |
| (L198Y) | (SEQ ID NO: 99) |
| | GTCGGGCCGCGATACAGCCGGCGGGAC |
| | (SEQ ID NO: 100) |
| Mutant 20 | GTCCCGCCGGCTGTGGCGCGGCCCGAC |
| (L198W) | (SEQ ID NO: 101) |
| | GTCGGGCCGCGCCACAGCCGGCGGGAC |
| | (SEQ ID NO: 102) |
| Mutant 21 | GTGCCCACCCCCCTGTTCGCGGCCGGCCGCGAC |
| (R101L) | (SEQ ID NO: 103) |
| | GTCGCGGCCGGCCGCGAACAGGGGGGTGGGCAC |
| | (SEQ ID NO: 104) |
| Mutant 22 | GTGCCCACCCCCACCTTCGCGGCCGGCCGCGAC |
| (R101T) | (SEQ ID NO: 105) |
| | GTCGCGGCCGGCCGCGAACAGGGGGGTGGGCAC |
| | (SEQ ID NO: 106) |
| Mutant 23 | CTTCGCGGCCGGCCTGGACCGCACGCTG |
| (R106L) | (SEQ ID NO: 107) |
| | CAGCGTGCGGTCCAGGCCGGCCGCGAAG |
| | (SEQ ID NO: 108) |
| Mutant 24 | CTTCGCGGCCGGCACCGACCGCACGCTG |
| (R106T) | (SEQ ID NO: 109) |
| | CAGCGTGCGGTCGGTGCCGGCCGCGAAG |
| | (SEQ ID NO: 110) |
| Mutant 25 | GCCGCGACCGCTTTCTGGCCCTGCTC |
| (T109F) | (SEQ ID NO: 111) |
| | GAGCAGGGCCAGAAAGCGGTCGCGGC |
| | (SEQ ID NO: 112) |
| Mutant 26 | GCCGCGACCGCATTCTGGCCCTGCTC |
| (T1091) | (SEQ ID NO: 113) |
| | GAGCAGGGCCAGAATGCGGTCGCGGC |
| | (SEQ ID NO: 114) |
| Mutant 27 | GCCGCGACCGCGTTCTGGCCCTGCTC |
| (T109V) | (SEQ ID NO: 115) |
| | GAGCAGGGCCAGAACGCGGTCGCGGC |
| | (SEQ ID NO: 116) |
| Mutant 52 | CGGCCGCGACCGCGATCTGGCCCTGCTC |
| (T109D) | (SEQ ID NO: 131) |
| | GAGCAGGGCCAGATCGCGGTCGCGGCCG |
| | (SEQ ID NO: 132) |
| Mutant 53 | CGGCCGCGACCGCCCGCTGGCCCTGCTC |
| (T109P) | (SEQ ID NO: 133) |
| | GAGCAGGGCCAGCGGGCGGTCGCGGCCG |
| | (SEQ ID NO: 134) |
| Mutant 30 | Using Mutant 13 as template DNA |
| (T109F L198F) | GCCGCGACCGCTTTCTGGCCCTGCTC |
| | (SEQ ID NO: 111) |
| | GAGCAGGGCCAGAAAGCGGTCGCGGC |
| | (SEQ ID NO: 112) |
| Mutant 31 | Using Mutant 25 as template DNA |
| (T109F I259A) | GTCGCTCGCCGTCGCGCTGCTGGTCGCC |
| | (SEQ ID NO: 83) |
| | GGCGACCAGCAGCGCGACGGCGAGCGAC |
| | (SEQ ID NO: 84) |
| Mutant 32 | Using Mutant 25 as template DNA |
| (T109F I259L) | GTCGCTCGCCGTCCTGCTGCTGGTCGCC |
| | (SEQ ID NO: 81) |
| | GGCGACCAGCAGCAGGACGGCGAGCGAC |
| | (SEQ ID NO: 82) |

This method used pHD05-SeuC10-SeuF08 as the template DNA and mutagenic primers. PCR reactions contained 12.5 μl of Phusion® High-Fidelity PCR Master Mix (New England Biolabs), ˜2 ng of template DNA, 1.25 μl DMSO and 0.5 UM of each forward and reverse primer. The reaction mix was topped up to a total volume of 25 μl with MilliQ®—H₂O. Amplification reactions were identical for all mutagenic reactions, with the exception of annealing temperatures which varied as follows: 70° C. for mutant 22 and mutant 24, and 64° C. for all other mutants. Reactions were performed on an Eppendorf Mastercycler Gradient with the following cycling conditions: 98° C. for 30 seconds, 16 cycles (98° C. for 30 seconds, annealing temperature for 1 minute, 72° C. for 8.5 minutes), 72° C. for 10 minutes. Amplifications were then subjected to Dpnl digestion.

### Dpnl Digestion

1 μl of Dpnl (20 U/μl; New England Biolabs), 1 μl Cutsmart buffer (New England Biolabs) was added to 8 μl of the PCR reaction. Unmutated template DNA was digested by incubating the reaction mix at room temperature for 5 minutes and then stored at 4° C. overnight.

### Cloning of Mutants

Dpnl reactions were used to introduce into E. coli DH5a cells by chemical transformation. 5 μl of each Dpnl reaction was combined with 50 μl chemically competent E. coli DH5a cells (Invitrogen). The transformation mixture was placed on ice for 30 minutes. Heat shock was performed for 30 seconds in a water bath at 42° C. and cells were subsequently chilled on ice for 5 minutes. After the addition of 950 μl of S.O.C Medium (Invitrogen), cells were incubated for 1 hour at 37° C. and 250 rpm in a New Brunswick Scientific Innova 4230. The transformation mixture was plated onto LB agar plates containing 50 μg/ml kanamycin. LB agar plates were allowed to incubate for 16 hours at 37° C. Individual colonies were picked and cultivated in a 24-well block containing 2.5 ml LB with 50 μg/ml kanamycin for 16 hours at 37° C. and 300 rpm. Recombinant plasmids were isolated from these cultures using the QIAprep 96 Turbo Miniprep Kit and analysed via DNA sequencing.

### DNA Sequencing and Analysis

DNA sequences of the cloned mutants and the reductase part of the pHD05 vector backbone were confirmed by Sanger sequencing at LGC Genomics (Germany).

### Example 3: Expression of Mutants

### Construction of the Recombinant Expression Strain

The strain E. coli Tuner (DE3) (Merck) was used as a host for recombinant expression of wild type and mutant variants of P450_{SeuC10}, ferredoxin_{SeuF08} and ferredoxin reductase_{SCF15A}. To construct this expression strain, expression plasmids were introduced into E. coli Tuner (DE3) by chemical transformation as described in Example 1.

### Expression of Recombinant Mutant SeuC10, SeuF08 and SCF15A

To prepare glycerol stocks of the expression strain, a streak of colonies were picked and inoculated into 5 ml LB containing 50 μg/ml kanamycin and cultivated at 37° C. and 250 rpm for approximately 16 hours. 500 μl of this culture were mixed with 500 μl of 50% glycerol (weight:vol) in cryovials and stored at −80° C.

Preculture: 5 ml of LB media supplemented with 50 μg/ml of kanamycin was inoculated with a loop scraped from a cryovial containing of E. coli Tuner (DE3) harboring the mutant expression plasmid. Cells were grown overnight at 37° C. and 250 rpm in a New Brunswick Scientific Innova 4230.

Seed: Into a 250 ml baffled flask, 50 ml of PCM8.1 media supplemented with 50 μg/ml of kanamycin was inoculated with the overnight preculture to an OD600 of 0.1 and incubated at 37° C. and 200 rpm until the end of the day.

The components of PCM8.1 were MgSO₄·7H₂O (0.49 gL⁻¹), Na₂HPO₄·7H₂O (6.7 gL⁻¹), KH₂PO₄ (3.4 gL⁻¹), NH₄Cl (2.68 gL⁻¹), Na₂SO₄ (0.71 gL⁻¹), arginine (0.2 gL⁻¹), histidine (0.15 gL⁻¹), lysine (0.2 gL⁻¹), phenylalanine (0.2 gL⁻¹), serine (0.2 gL⁻¹), threonine (0.2 gL⁻¹), methionine (0.2 gL⁻¹), monosodium glutamate (8 gL⁻¹), glucose (0.5 gL⁻¹), glycerol (10 gL⁻¹) and a 1000-fold diluted trace element solution with FeCl₃ (81.1 gL⁻¹), CaCl₂·6H₂O (4.38 gL⁻¹), MnCl₂·4H₂O (1.98 gL⁻¹), ZnSO₄·7H₂O (2.88 gL⁻¹), CoCl₂·6H₂O (0.48 gL⁻¹), CuCl₂·2H₂O (0.34 gL⁻¹), NiCl₂·6H₂O (0.48 gL⁻¹), Na₂MoO₄·2H₂O (0.48 gL⁻¹), Na₂SeO3 (0.35 gL⁻¹), and H₃BO₃ (0.12 gL⁻¹).

Production: At the end of the day, a 1 L baffled flask containing 200 mL of PCM8. 1 media supplemented with 50 μg/ml of kanamycin, 23.8 μg/ml of IPTG, 320 μg/ml of 5′-aminolevulinic acid and 55 μg/ml of FeSO₄·7H₂O was inoculated with the seed cultures to an OD of 0.6. The induced production cultures were incubated at 27° C. and 200 rpm until the cultures had reached stationary phase (approximately 16-20 hours). The cultures were harvested by centrifugation at 3,000 rpm for 15 minutes. The pellets were washed with 30 ml of wash buffer (isotonic 0.85% NaCl with 5% glycerol) and transferred into a fresh 50 ml falcon tube. The cells were further centrifuged at 4,000 rpm for 25-35 minutes and the pellet was stored at −20° C. for processing.

### Example 4: Extraction & Processing of Enzyme Materials

Suspended cell pellets were provided as described in Example 2, containing recombinant P450, ferredoxin and ferredoxin reductase in 100 mM potassium phosphate buffer pH 8.0, 5 mM MgCl₂, 0.5 mM TCEP, and 1 mM PMSF in a ratio of 1.0 ml of buffer per 0.3 g of cells. Benzonase nuclease (Sigma-Aldrich, UK) was added at a volume of 0.5 μl to all samples. Lysed cells were produced by high pressure disruption using three cycles of 30 KPsi. Lysed material was centrifuged at 38,800×g for 40 minutes)(4° C. The supernatant containing recombinant P450, ferredoxin and ferredoxin reductase was either used immediately for the desired reaction or dispensed into glass vials (typically 0.5 ml per 2 ml vial), frozen and lyophilised using an Edwards Supermodulyo Freeze-dryer before being sealed under vacuum and stored in a standard laboratory freezer at −20° C. until required for use.

Measurements of the concentration of cytochrome P450 were performed according to the method of Omura and Sato et al. (J. Biol. Chem., 239. 1964, 2370). The cytochrome P450 concentration of cell-free extracts of induced E. coli Tuner (DE3) cells harbouring pHD05-SeuC10-SeuF08 expression plasmid and variants ranged between 17.3 and 39 UM, except for mutant 16 (L198N) which was poorly expressed.

### Example 5: Oxidase Activity/Spectrum Testing

Lyophilised material of recombinant P450, ferredoxin and ferredoxin reductase proteins was made as described in Example 4 and reconstituted in high purity water to the original volume. Biocatalysis was performed with shaking incubation at 27° C. in the following conditions: 100 mM potassium phosphate pH 8.0, 5 mM MgCl₂ (both present in the reconstituted enzyme preparation) and 0.1 mg/ml substrate compound such as ambroxide (Sigma-Aldrich, UK), carbamazepine (Sigma-Aldrich, UK), diclofenac (Sigma-Aldrich, UK), tivantinib (MedChemtronica, Sweden), clarithromycin (Tokyo Chemical Industry, Japan), sclareol (Sigma-Aldrich, UK), celecoxib (LC Laboratories, USA), buparvaquone (Sigma-Aldrich, UK), BIRB796 (MedChemExpress, USA), vanoxerine (CiVentiChem, USA), valsartan (Tokyo Chemical Industry, Japan), ruxolitinib (LC Laboratories, USA), perindopril (Selleckchem, USA), solifenacin (Key Organics, UK), 7-ethoxycoumarin (Tokyo Chemical Industry, Japan), tofacitinib (LC Laboratories, USA), ebastine (Key Organics, UK), efavirenz (Key Organics, UK), propanalol (Sigma-Aldrich, UK) and tolbutamide (Key Organics, UK).

Concentrations of P450, ferredoxin and ferredoxin reductase were as extracted (Example 4). Reactions were initiated by addition of 10× stock of cofactor mixture (50 mM G6P, 10 mM NADP+, 10 UN/ml G6PDH) to provide a final volume of e.g., 10 μl to 90 μl for 100 μl total reaction volumes. After 16-20 hours, reactions were extracted with an equal volume of acetonitrile, centrifuged to remove precipitated proteins and conversion assessed by UPLC-MS analysis.

UPLC Data were Obtained as Follows:
Column: Acquity UPLC BEH Shield RP18 1.7 μm 2.1 mm i.d. 50 mm length
Solvents: H₂O, B: Acetonitrile, both with 0.1% Formic acid
Flow rate: 1.0 ml/min
Detector: Waters Acquity UPLC PDA (UV-Vis detection) and Waters Acquity UPLC QDA (MS)

To confirm the identities of reaction products where known, their chromatographic retention time, mass and ultraviolet spectra were compared with those of authentic metabolite standards.

Representative results for the % conversions of ambroxide to oxidised (+16 Da) products for single point mutants at position T109 are shown in Table 2 below. Mutants at positions other than T109 did not demonstrate capabilities to produce new products of interest from ambroxide.

TABLE 2
Results (% conversions measured by relative MS peak areas) for testing with
lyophilised material containing P450_{SeuC10} T109 mutants co-expressed with
ferredoxin_{SeuF08} and ferredoxin reductase_{SCF15A} against ambroxide.

| | | P450 | Ambroxide | +16 Da | +16 Da | +16 Da | |
|---|---|---|---|---|---|---|---|
| | | Concentration | (2.22 min) | (1.50 min) | (2.04 min) | (1.60 min) | Average % |
| P450 | Mutation | (μM) | 237 m/z | 253 m/z | 253 m/z | 253 m/z | conversion |
| SeuC10 | N/A | 28.8 | 22.8 | 72.2 | 0.0 | 5.0 | 77.2 |
| SeuC10M1 | T109R | 17.3 | 59.3 | 40.7 | 0.0 | 0.0 | 40.7 |
| SeuC10M2 | T109A | 32 | 6.8 | 85.1 | 0.0 | 7.0 | 93.2 |
| SeuC10M25 | T109F | 28.9 | 18.9 | 52.0 | 13.9 | 15.2 | 81.1 |
| SeuC10M26 | T109I | 29.4 | 41.7 | 58.3 | 0.0 | 0.0 | 58.3 |
| SeuC10M27 | T109V | 32.8 | 38.2 | 58.6 | 2.3 | 0.9 | 61.7 |

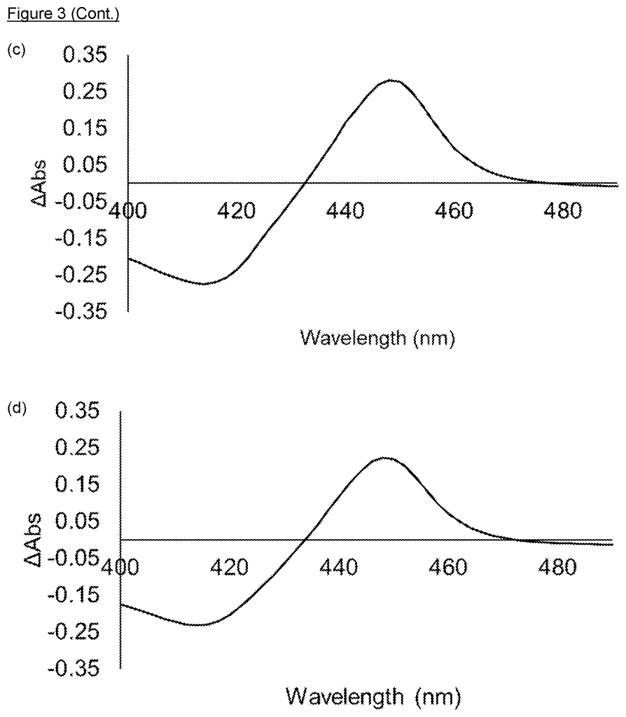

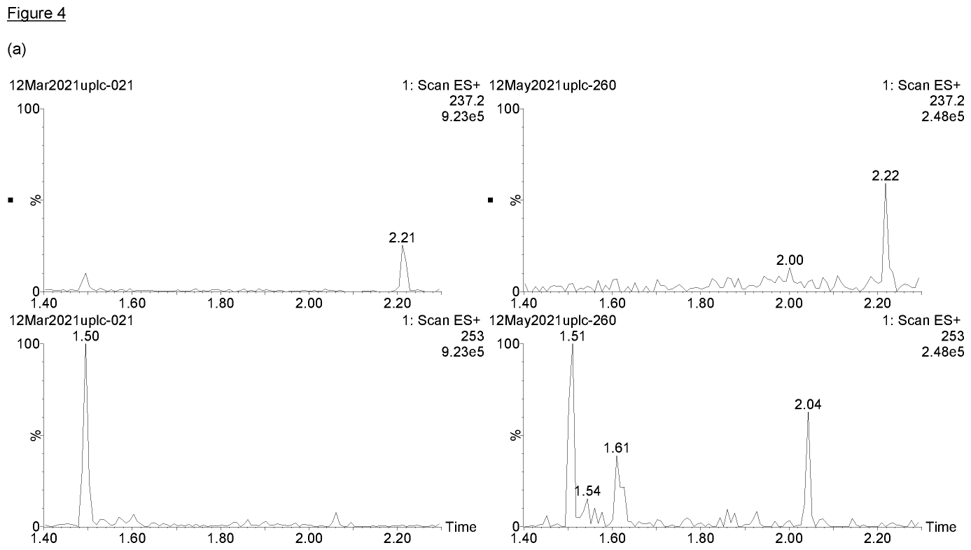

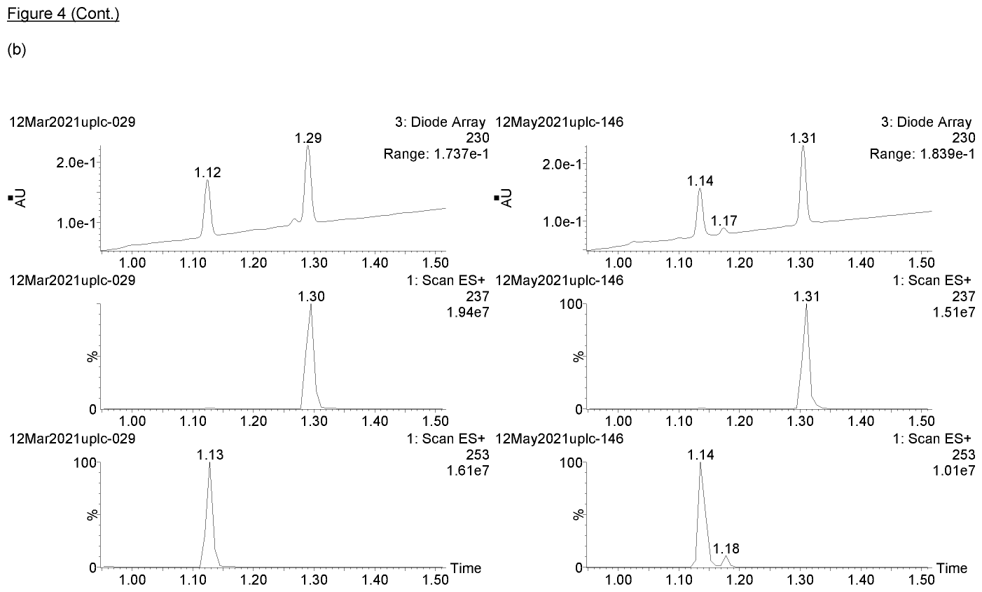

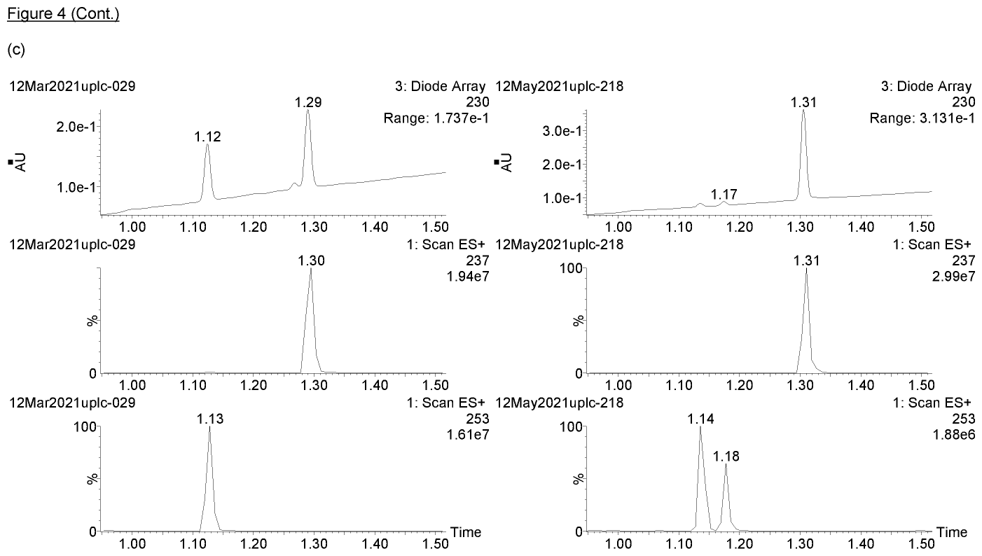

SeuC10M25 is clearly noteworthy in producing the highest yields of the products at 1.61 and 2.04 minutes, and the chromatographic analysis of this reaction is shown in FIG. 4a. SeuC10 and the other mutants, by comparison, produce the product at 1.50 as the major product with small amounts of the product at 1.61 minutes.

Representative results for the % conversions of carbamazepine to oxidised (+16 Da) products for single point mutants at position L198 are shown in Table 3 below. Mutants at positions other than L198 did not demonstrate capabilities to produce new products of interest from carbamazepine.

TABLE 3
Results (% conversions measured by relative MS peak areas) for testing with
lyophilised material containing P450_{SeuC10} L198 mutants co-expressed with
ferredoxin_{SeuF08} and ferredoxin reductase_{SCF15A} against carbamazepine.

| | | P450 | Parent | +16 Da | +16 Da | |
|---|---|---|---|---|---|---|
| | | Concentration | (1.31 min) | (1.14 min) | (1.18 min) | Average % |
| P450 | Mutation | (μM) | 237 m/z | 253 m/z | 253 m/z | conversion |
| SeuC10 | N/A | 28.8 | 56.4 | 43.6 | 0.0 | 43.6 |
| SeuC10M8 | L198I | 27.2 | 64.7 | 35.3 | 0.0 | 35.3 |
| SeuC10M9 | L198A | 22.4 | 83.1 | 15.8 | 1.1 | 16.9 |
| SeuC10M12 | L198M | 39.0 | 59.5 | 40.5 | 0.0 | 40.5 |
| SeuC10M13 | L198F | 34.3 | 54.6 | 42.3 | 3.1 | 45.4 |
| SeuC10M14 | L198V | 36.9 | 56.3 | 43.7 | 0.0 | 43.7 |
| SeuC10M16 | L198N | 2.8 | 98.1 | 1.9 | 0.0 | 1.9 |
| SeuC10M17 | L198D | 26.1 | 96.8 | 3.2 | 0.0 | 3.2 |
| SeuC10M18 | L198H | 38.1 | 85.4 | 14.6 | 0.0 | 14.6 |
| SeuC10M19 | L198Y | 29.5 | 96.1 | 3.9 | 0.0 | 3.8 |
| SeuC10M20 | L198W | 27.9 | 89.7 | 6.8 | 3.5 | 10.3 |

SeuC10 itself and most of the enzymes with mutations at this position produce carbamazepine epoxide as the only reaction product at a retention time of 1.14 minutes. However, SeuC10M9, SeuC10M13 and SeuC10M20 produce a new product with a retention time of 1.18 minutes, and the chromatograms for the SeuC10M13 and SeuC10M20 reactions are shown in FIGS. 4b and 4c, respectively. The new product has been identified as 2-hydroxycarbamazepine.

Representative results for the % conversions of various substrates to products by enzymes with mutations at position T109 or L198 and double mutants at T109 and 1129/L198 are shown in Table 4 below.

TABLE 4
Average % conversion (% conversions measured by UPLC-MS UV or MS peak areas) using lyophilised material containing
P450_{SeuC10} mutants co-expressed with ferredoxin_{SeuF08} and ferredoxin reductase_{SCF15A} against substrates as indicated,
compared to conversion rates of the wild type (WT) enzyme. These assays were conducted in two batches as indicated.

| | Batch 1 | | | | | | Batch 2 | | | | | | |
|---|---|---|---|---|---|---|---|---|---|---|---|---|---|
| | Substrate | | | | | | | | | | | | |
| | M2 | M9 | M13 | M20 | M25 | WT | M15 | M30 | M31 | M32 | M52 | M53 | WT |
| | Mutation(s) | | | | | | | | | | | | |
| | | | | | | | | L198F | T109F | T109F | | | |
| | T109A | L198A | L198F | L198W | T109F | | L198T | T109F | I259A | I259L | T109D | T109P | |
| Sclareol | 36.5 | 75.2 | 41.3 | 0 | 55.7 | 38.5 | 61 | 31 | 1.3 | 84.5 | 25.4 | 52.7 | 35.5 |
| Celecoxib | 24.7 | 2.6 | 26.8 | 6.9 | 10.4 | 29.4 | 7.2 | 21.6 | 0.4 | 35.6 | 29.7 | 39.1 | 22.2 |
| Buparvaquone | 28.7 | 1.2 | 39.1 | 1.7 | 19.4 | 23.5 | 0 | nt | 0 | 31.5 | 24.8 | 54.7 | 32.9 |
| BIRB796 | 29.5 | 10 | 54.6 | 45.3 | 13.9 | 83.1 | 34.4 | 38.5 | 0 | 90.6 | 13 | 36.3 | 78.8 |
| Vanoxerine | 87.2 | 23 | 29.4 | 29.4 | 58 | 75.8 | 41 | 79.1 | 11.4 | 88.8 | 65 | 44.5 | 65.8 |
| Valsartan | 17.6 | 6.9 | 12 | 25 | 15.5 | 20.1 | 20.9 | 17.1 | 0.6 | 66.8 | 16.1 | 14.3 | 20.8 |
| Ruxolitinib | 92.8 | 55.1 | 98.1 | 42.7 | 79.8 | 80.9 | 100 | 99.1 | 46.9 | 99.2 | 87.7 | 98.6 | 98.6 |
| Perindopril | 30.7 | 15.7 | 38.2 | 16.5 | 23.5 | 30.9 | 35.4 | 61.4 | 14.6 | 45.6 | 37.9 | 46.5 | 32.7 |
| Solifenacin | 7.2 | 0.8 | 0.8 | 0.6 | 2.9 | 5.3 | 6 | 3 | 2.2 | 25.9 | 17.2 | 33.6 | 7.2 |
| 7- | 32.4 | 10.7 | 31.2 | 11.4 | 17.5 | 22.9 | 14.7 | 38 | 2.9 | 20.2 | 17.3 | 67.2 | 23.3 |
| ethoxycoumarin | | | | | | | | | | | | | |
| Tofacitinib | 47.1 | 6.2 | 32.2 | 5.9 | 21.4 | 36.7 | 32.3 | 33.3 | 8.2 | 40.8 | 38.2 | 67.5 | 51.6 |
| Ebastine | 6.8 | 25.1 | 14.7 | 19.2 | 12.7 | 8.8 | 0.5 | 9.7 | 0 | 10.4 | 3.1 | 17.4 | 4.5 |
| Efavirenz | 0.9 | 3.1 | 0 | 0 | 1.5 | 0 | 4.3 | 0 | 4.1 | 1.2 | 0 | 1.8 | 0 |
| Clarithromycin | 0 | 0 | 0 | 0 | 0 | 0 | 0 | 0.9 | 0.7 | 23.3 | 1 | nt | 0 |
| Propanalol | 0 | 0 | 0 | 0 | 0 | 0 | 0 | 0 | 0 | 1.6 | 0 | nt | 0 |
| Tolbutamide | 52.3 | 14.7 | 27.9 | 13.7 | 28.2 | 32.2 | 28.3 | 35.7 | 2.9 | 56.1 | 26.3 | 77.6 | 37.7 |

The results in Table 4 show that the mutants increased the conversion of various substrates and/or achieved conversions not observed with the wild-type enzyme. Of note, M2 significantly increased conversion of tolbutamide; M9 significantly increased conversion of sclareol and ebastine; M30 significantly increased conversion of perindopril, M32 significantly increased conversion of sclareol, vanoxerine and valsartan; and M53 significantly increased conversion of buparvaquone, solifenacin, 7-ethoxycoumarin and tolbutamide. Several mutants also achieved conversion of substrates which could not be converted by the wild-type enzyme, particularly M32 (efavirenz, clarithromycin and propanalol).

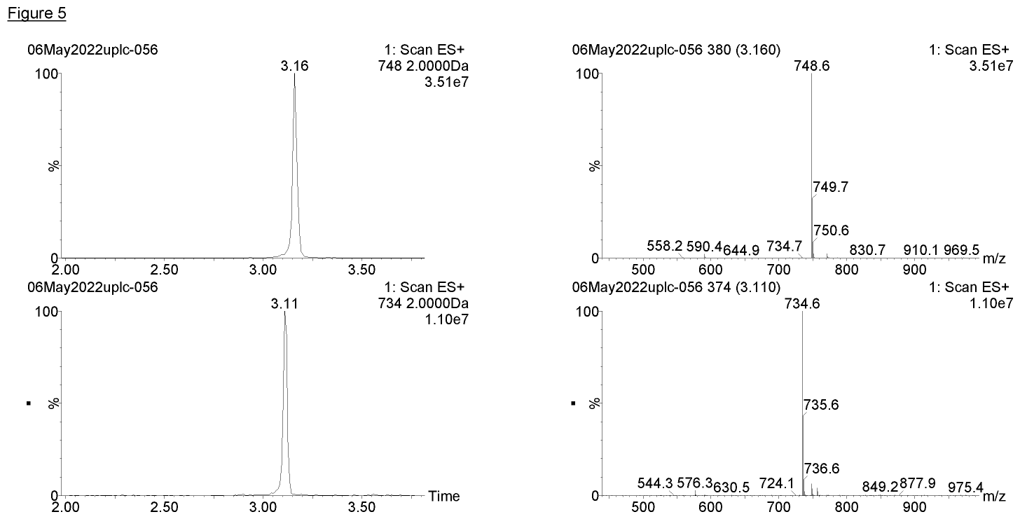

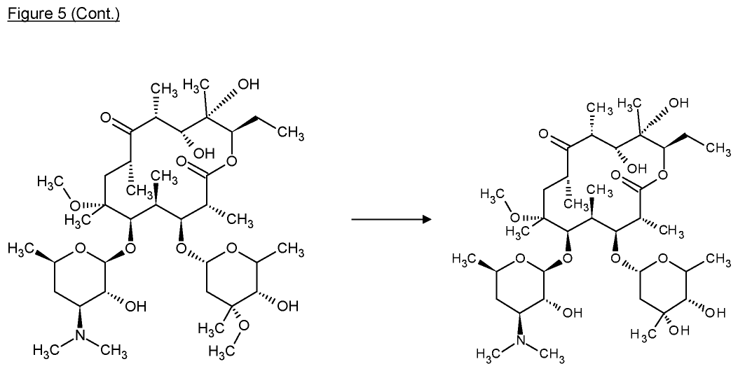

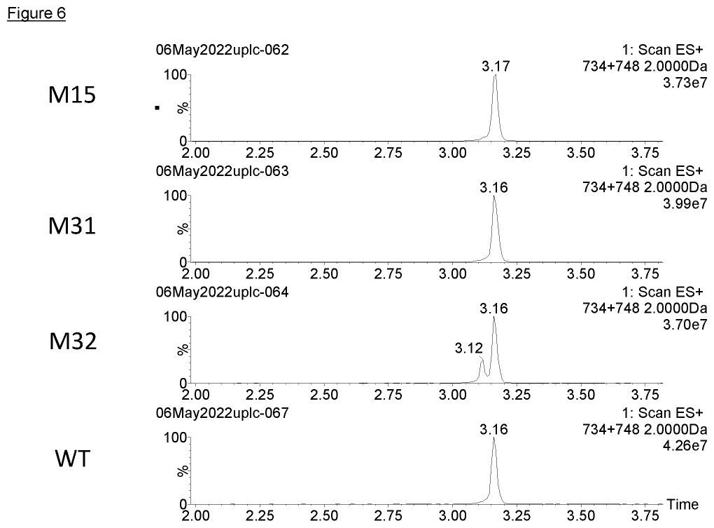

The chromatographic analysis of the products resulting from the reaction of M32 with clarithromycin revealed the production of a new product at 3.12 minutes (FIGS. 5 and 6). This product was not observed using M15 or M31. The product is 3″-O-demethylclarithromycin, as shown in FIG. 5.

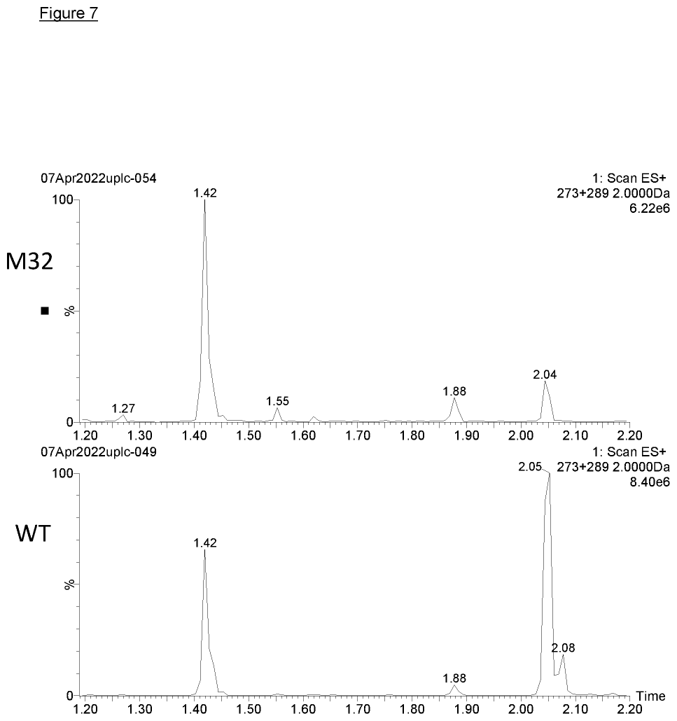

M32 also increased production of the product at 1.42 minutes and generated a new product at 1.55 minutes when using sclareol as the substrate (FIG. 7).

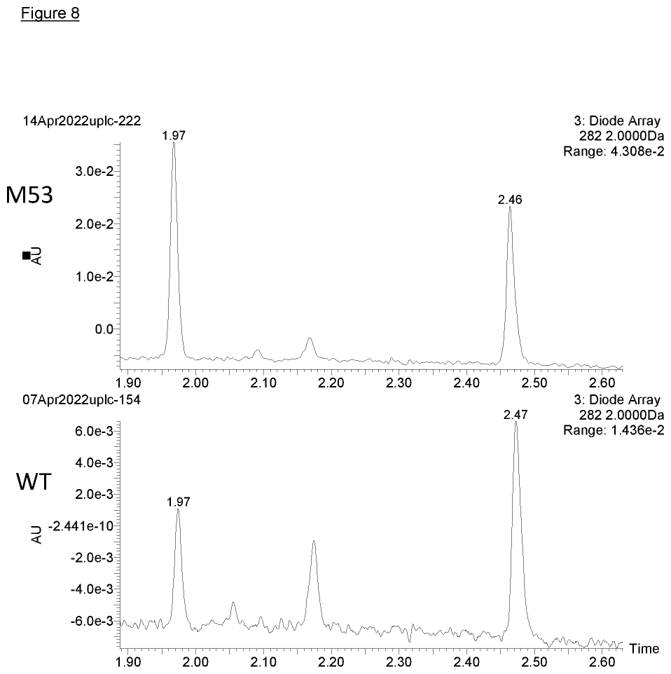

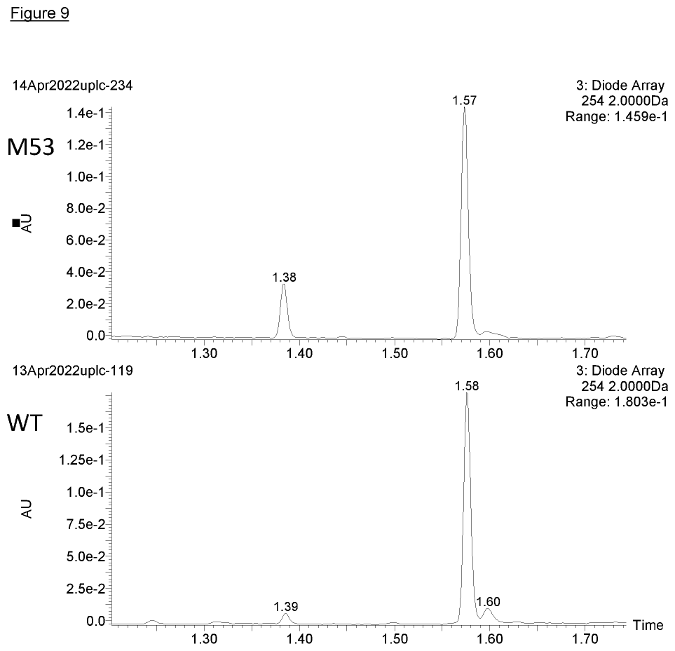

M53 increased production of the product at 1.97 minutes, which results from the hydroxylation of a t-butyl methyl group, when using buparvaquone as the substrate (FIG. 8) and also increased production of the product at 1.38 minutes when using ebastine as the substrate (FIG. 9).

### Sequence Listing Sequences:

SEQ ID NO: 1-P450 SeuC10 and ferrodoxin SeuF08 operon from Streptomyces eurythermus NRRL 2539
SEQ ID NO: 2-P450 SeuC10 wild type from Streptomyces eurythermus NRRL 2539, protein
SEQ ID NO: 3-P450 SeuC10 from Streptomyces eurythermus NRRL 2539 with unspecified residues at positions 109 & 198, protein
SEQ ID NO: 4-ferrodoxin SeuF08 from Streptomyces eurythermus NRRL 2539, protein
SEQ ID NOs: 5 and 6-SeuC10M1-M1 mutant, encoding and protein sequence
SEQ ID NOs: 7 and 8-SeuC10M2-M2 mutant, encoding and protein sequence
SEQ ID NOs: 9 and 10-SeuC10M3-M3 mutant, encoding and protein sequence
SEQ ID NOs: 11 and 12-SeuC10M4-M4 mutant, encoding and protein sequence
SEQ ID NOs: 13 and 14-SeuC10M5-M5 mutant, encoding and protein sequence
SEQ ID NOs: 15 and 16-SeuC10M6-M6 mutant, encoding and protein sequence
SEQ ID NOs: 17 and 18-SeuC10M7-M7 mutant, encoding and protein sequence
SEQ ID NOs: 19 and 20-SeuC10M8-M8 mutant, encoding and protein sequence
SEQ ID NOs: 21 and 22-SeuC10M9-M9 mutant, encoding and protein sequence
SEQ ID NOs: 23 and 24-SeuC10M10-M10 mutant, encoding and protein sequence
SEQ ID NOs: 25 and 26-SeuC10M11-M11 mutant, encoding and protein sequence
SEQ ID NOs: 27 and 28-SeuC10M12-M12 mutant, encoding and protein sequence
SEQ ID NOs: 29 and 30-SeuC10M13-M13 mutant, encoding and protein sequence
SEQ ID NOs: 31 and 32-SeuC10M14-M14 mutant, encoding and protein sequence
SEQ ID NOs: 33 and 34-SeuC10M15-M15 mutant, encoding and protein sequence
SEQ ID NOs: 35 and 36-SeuC10M16-M16 mutant, encoding and protein sequence
SEQ ID NOs: 37 and 38-SeuC10M17-M17 mutant, encoding and protein sequence
SEQ ID NOs: 39 and 40-SeuC10M18-M18 mutant, encoding and protein sequence
SEQ ID NOs: 41 and 42-SeuC10M19-M19 mutant, encoding and protein sequence
SEQ ID NOs: 43 and 44-SeuC10M20-M20 mutant, encoding and protein sequence
SEQ ID NOs: 45 and 46-SeuC10M21-M21 mutant, encoding and protein sequence
SEQ ID NOs: 47 and 48-SeuC10M22-M22 mutant, encoding and protein sequence
SEQ ID NOs: 49 and 50-SeuC10M23-M23 mutant, encoding and protein sequence
SEQ ID NOs: 51 and 52-SeuC10M24-M24 mutant, encoding and protein sequence
SEQ ID NOs: 53 and 54-SeuC10M25-M25 mutant, encoding and protein sequence
SEQ ID NOs: 55 and 56-SeuC10M26-M26 mutant, encoding and protein sequence
SEQ ID NOs: 57 and 58-SeuC10M27-M27 mutant, encoding and protein sequence
SEQ ID NOs: 59-62-Primers from Example 1
SEQ ID NOs: 63-116-Primers from Example 2
SEQ ID NO: 117-P450 SeuC10 wild type from Streptomyces eurythermus NRRL 2539, encoding sequence
SEQ ID NO: 118-ferrodoxin SeuF08 from Streptomyces eurythermus NRRL 2539, encoding sequence
SEQ ID NOs: 119 and 120-ferrodoxin reductase Scf15A from Streptomyces coelicolor A3(2), protein and encoding sequence
SEQ ID NOs: 121 and 122-SeuC10M52-M52 mutant, encoding and protein sequence
SEQ ID NOs: 123 and 124-SeuC10M53-M53 mutant, encoding and protein sequence
SEQ ID NOs: 125 and 126-SeuC10M30-M30 mutant, encoding and protein sequence
SEQ ID NOs: 127 and 128-SeuC10M31-M31 mutant, encoding and protein sequence
SEQ ID NOs: 129 and 130-SeuC10M32-M32 mutant, encoding and protein sequence
SEQ ID NOs: 131-134-Primers from Example 2

1. An enzyme with cytochrome P450 activity and comprising the amino acid sequence set forth in SEQ ID NO: 3, or an amino acid sequence having at least 80% identity thereto, wherein the amino acid at the position corresponding to position 109 of SEQ ID NO: 3 is not threonine, and/or the amino acid at the position corresponding to position 198 of SEQ ID NO: 3 is not leucine.

2. The enzyme of claim 1, wherein the amino acid at the position corresponding to position 109 of SEQ ID NO: 3 is selected from phenylalanine, alanine, valine and proline, preferably wherein the amino acid is phenylalanine.

3. The enzyme of claim 2, comprising the amino acid sequence set forth in SEQ ID NO: 54 or an amino acid sequence having at least 80% identity thereto, preferably at least 95% identity thereto.

4. The enzyme of any one of claims 1 to 3, wherein
a) the amino acid at the position corresponding to position 109 of SEQ ID NO: 3 is phenylalanine, and
b) the amino acid at the position corresponding to position 259 of SEQ ID NO: 3 is not isoleucine and/or the amino acid at the position corresponding to position 198 of SEQ ID NO: 3 is not leucine.

5. The enzyme of claim 4, wherein
a) the amino acid at the position corresponding to position 109 of SEQ ID NO: 3 is phenylalanine and the amino acid at the position corresponding to position 259 of SEQ ID NO: 3 is leucine; or
b) the amino acid at the position corresponding to position 109 of SEQ ID NO: 3 is phenylalanine and the amino acid at the position corresponding to position 198 of SEQ ID NO: 3 is phenylalanine.

6. The enzyme of claim 5, comprising the amino acid sequence set forth in SEQ ID NO: 126 or 130 or an amino acid sequence having at least 80% identity thereto, preferably at least 95% identity thereto.

7. The enzyme of any one of claims 1 to 6, wherein the amino acid at the position corresponding to position 198 of SEQ ID NO: 3 is alanine, phenylalanine or tryptophan.

8. The enzyme of claim 7, comprising the amino acid sequence set forth in SEQ ID NO: 22, 30 or 44 or an amino acid sequence having at least 80% identity thereto, preferably at least 95% identity thereto.

9. The enzyme of any one of claims 1 to 3, wherein
a) the amino acid at the position corresponding to position 198 of SEQ ID NO: 3 is phenylalanine, and
b) the amino acid at the position corresponding to position 109 of SEQ ID NO: 3 is not threonine.

10. A nucleic acid molecule encoding an enzyme with cytochrome P450 activity as defined in any one of claims 1 to 9.

11. A recombinant construct comprising the nucleic acid molecule of claim 10 operatively linked to an expression control sequence.

12. A vector comprising the nucleic acid molecule of claim 10 or the recombinant construct of claim 11.

13. A microorganism comprising the nucleic acid molecule of claim 10, the recombinant construct of claim 11 or the vector of claim 12.

14. The microorganism of claim 13, wherein the microorganism is Escherichia coli.

15. A kit comprising:
i) an enzyme with cytochrome P450 activity as defined in any one of claims 1 to 9; and/or
ii) a microorganism that expresses an enzyme with cytochrome P450 activity as defined in any one of claims 1 to 9, preferably a microorganism as defined in claim 13 or 14, or a lysate thereof; and/or
iii) a nucleic acid molecule as defined in claim 10, a recombinant construct as defined in claim 11 or a vector as defined in claim 12.

16. The kit of claim 15, further comprising a reducing agent, preferably a ferredoxin reductase and a ferredoxin.

17. The kit of claim 15 or 16, further comprising a buffer.

18. The kit of any one of claims 15 to 17, wherein:
(i) the kit further comprises at least one other cytochrome P450 enzyme; and/or
(ii) the microorganism further expresses at least one other cytochrome P450 enzyme; and/or
(iii) the kit further comprises at least one other microorganism expressing another cytochrome P450 enzyme, or a lysate thereof.

19. The kit of any one of claims 15 to 18, wherein the enzyme, microorganism or lysate, or nucleic acid molecule, recombinant construct or vector is lyophilised and/or vacuum sealed.

20. A method of producing an oxidised organic compound, comprising contacting the organic compound with the enzyme of any one of claims 1 to 9 to oxidise said organic compound.

21. Use of the enzyme of any one of claims 1 to 9 to oxidise an organic compound.

22. The method of claim 20 or the use of claim 21, wherein the oxidation is hydroxylation, epoxidation, carboxylation or dealkylation, preferably demethylation.

23. The method or use of any one of claims 20 to 22, wherein the organic compound is an aromatic compound and/or a terpenoid, preferably an aliphatic terpenoid.

24. The method or use of any one of claims 20 to 23, wherein the enzyme catalyzes hydroxylation of an aromatic ring on said organic compound.

25. The method or use of any one of claims 20 to 24, wherein the enzyme is used in combination with reductase components or other reducing agents, preferably with ferredoxin and ferredoxin reductase components.

26. The method or use of any one of claims 20 to 25, wherein the enzyme is in a purified form, a part-purified form, a cell lysate or a recombinant host cell.

27. The method or use of claim 26, wherein the enzyme is in a recombinant host cell, and said method or use comprises dosing the cells with the organic compound to be oxidised, and optionally further comprises subsequently harvesting the host cell and isolating the oxidised compound.

28. The method or use of any one of claims 20 to 27 wherein said method further comprises the step of harvesting, collecting, isolating, enriching, purifying and/or processing the oxidised compound.

29. A method of producing an enzyme with cytochrome P450 activity as defined in any one of claims 1 to 9, comprising introducing a nucleic acid molecule as defined in claim 10, a recombinant construct as defined in claim 11 or a vector as defined in claim 12 into a microorganism, and expressing the cytochrome P450 enzyme in the microorganism, and optionally purifying the enzyme.
